(19) United States
(12) Patent Application Publication
(10) Pub. No.: US 2026/0285557 A1
(43) Pub. Date: Sep. 24, 2026

(54) ACTIVE PRESSURE MONITORING CLOSURE

(71) Applicant: Mickel Roach, Safety Harbor, FL (US)

(72) Inventor: Mickel Roach, Safety Harbor, FL (US)

(21) Appl. No.: 19/687,474

(22) Filed: May 26, 2026

Related U.S. Application Data
(63) Continuation-in-part of application No. 17/664,785, filed on May 24, 2022, now Pat. No. 12,637,641.

(51) Int. Cl. B65D 51/16; B65D 1/02; B65D 41/04; C12C 13/10

(52) U.S. Cl. CPC B65D 51/1644; B65D 1/0207; B65D 1/0246; B65D 41/04; B65D 2205/02; C12C 13/10

(57) ABSTRACT

Apparatus and associated methods relate to an umbrella valve having a retention module positioned above a diaphragm plane. In an illustrative embodiment, the umbrella valve may include an umbrella valve body extending along a longitudinal axis. The body may, for example, include a diaphragm extending outwards away from the longitudinal axis and towards a proximal end. The body may, for example, include a retention module configured to engage a first side of a surface when disposed through an aperture. An outer circumference of the diaphragm may, for example, define a perimeter in a first plane. The retention module may, for example, rest above the first plane in a non-deployed mode such that, in a deployed mode, the retention module causes the umbrella valve to be preloaded with a predetermined tension. Various embodiments may advantageously enable a high pressure umbrella valve in a thin wall.

### CROSS-REFERENCE TO RELATED APPLICATIONS

This application is a Continuation-in-Part and claims the benefit of U.S. application Ser. No. 17/664,785, titled “Active Carbonation Pressure Monitoring Cap,” filed by Mickel Roach on May 24, 2022. This application incorporates the entire contents of the foregoing application herein by reference.

This application shares a common inventor and/or subject matter with the following applications:
WO patent application serial no. PCT/US23/23242, filed May 23, 2023 (naming inventor(s) including ROACH, Mickel) and titled “Active Carbonation Pressure Monitoring Cap;”
AU patent application serial no. 2023275586, filed May 23, 2023 (naming inventor(s) including ROACH, Mickel) and titled “Active Carbonation Pressure Monitoring Cap;”
BR patent application serial no. BR 11 2024 024362 1, filed May 23, 2023 (naming inventor(s) including ROACH, Mickel) and titled “Active Carbonation Pressure Monitoring Cap;”
CA patent application serial no. 3255567, filed May 23, 2023 (naming inventor(s) including ROACH, Mickel) and titled “Active Carbonation Pressure Monitoring Cap;”
CN patent application serial no. 202380041972, filed May 23, 2023 (naming inventor(s) including ROACH, Mickel) and titled “Active Carbonation Pressure Monitoring Cap;”
EP patent application serial no. 23733498, filed May 23, 2023 (naming inventor(s) including ROACH, Mickel) and titled “Active Carbonation Pressure Monitoring Cap;”
IN patent application serial no. 202447090710, filed May 23, 2023 (naming inventor(s) including ROACH, Mickel) and titled “Active Carbonation Pressure Monitoring Cap;”
JP patent application serial no. 2024569083, filed May 23, 2023 (naming inventor(s) including ROACH, Mickel) and titled “Active Carbonation Pressure Monitoring Cap;”
KR patent application serial no. 10-2024-7042401, filed May 23, 2023 (naming inventor(s) including ROACH, Mickel) and titled “Active Carbonation Pressure Monitoring Cap;”
MX patent application serial no. MX/a/2024/014441, filed Nov. 21, 2024 (naming inventor(s) including ROACH, Mickel) and titled “Active Carbonation Pressure Monitoring Cap;” and
RU patent application serial no. 2024137850, filed May 23, 2023 (naming inventor(s) including ROACH, Mickel) and titled “Active Carbonation Pressure Monitoring Cap.”

This application incorporates the entire contents of the foregoing applications herein by reference.

### TECHNICAL FIELD

Various embodiments relate generally to pressure management in a closed volume(s).

### BACKGROUND

Humans have used fermentation to produce foodstuffs and beverages since thousands of years ago. For example, some food preservation process may use fermentation in a process that produces lactic acid. Some examples of fermented food are pickled cucumbers, kombucha, kimchi, and yogurt. In some examples, fermentation may also be used for producing alcoholic beverages such as wine and beer.

In some examples, fermentation is a metabolic process that produces chemical changes in organic substrates through the action of enzymes. In food production, for example, fermentation may refer to a process in which the activity of micro-organisms (e.g., probiotic culture, yeast, and/or inoculant) brings about a desirable change to a foodstuff or beverage.

Carbonated drinks may be made from a fermentation process where yeast or other fermentation culture is added to a fermenting liquid (e.g., juice) to cause carbonation. In some examples, carbonation may occur when yeasts consume sugar in a juice and output carbon dioxide. When the carbon dioxide is contained, for example, in a bottle, the carbon dioxide may build up in the bottle and turns into bubbles in the fermenting liquid. As a result, gas pressure may vary during the fermentation process.

### SUMMARY

Apparatus and associated methods relate to an umbrella valve having a retention module positioned above a diaphragm plane. In an illustrative embodiment, the umbrella valve may include an umbrella valve body extending along a longitudinal axis. The body may, for example, include a diaphragm extending outwards away from the longitudinal axis and towards a proximal end. The body may, for example, include a retention module configured to engage a first side of a surface when disposed through an aperture. An outer circumference of the diaphragm may, for example, define a perimeter in a first plane. The retention module may, for example, rest above the first plane in a non-deployed mode such that, in a deployed mode, the retention module causes the umbrella valve to be preloaded with a predetermined tension. Various embodiments may advantageously enable a high pressure umbrella valve in a thin wall.

Apparatus and associated methods relate to a fermentation monitoring apparatus configured to generate a visual indicia in a fluid well in response to pressure inside a vessel exceeding a predetermined pressure. In an illustrative example, the fermentation monitoring apparatus may include a vessel cap that includes a fluid well holding a quantity of fluid, and at least one fluid communication channel. The fluid well, for example, may receive gas released from the channel via the at least one fluid communication channel when the vessel cap is coupled to a first end of a vessel to define a chamber and a gas pressure in the chamber exceeds a predetermined threshold. Various embodiments may advantageously generate a visual indicium of gas bubbles in the quantity of fluid indicating to a user that the gas pressure in the chamber has exceeded the predetermined threshold.

Apparatus and associated methods relate to a pressure-management adapter having an adapter body with a ball-lock-compatible coupling interface, a pressure pathway extending through the adapter body, and an umbrella valve mounted at an aperture in fluid communication with the pressure pathway. In an illustrative example, the adapter body may include a gas-post or liquid-post ball-lock interface configured to couple to existing ball-lock fittings on growlers, mini-kegs, and/or kegging hardware. The umbrella valve, for example, may remain closed below a threshold pressure and may open above the threshold pressure to release gas from the pressure pathway. In some examples, gas released through the umbrella valve may be vented to atmosphere through a vent outlet in the adapter body. In some implementations, the adapter body may include a fluid well positioned to receive gas released through the umbrella valve, and a viewing fluid held in the fluid well may form visible bubbles when gas is released. In some examples, the umbrella valve may be removably received in the aperture and may be replaced with a second umbrella valve associated with a different threshold pressure. In various implementations, the adapter may be used during at least one of: purging, carbonation, dispensing, fermentation pressure control, and/or storage pressure management. Various embodiments may allow threshold-based pressure management to be applied to vessels and hardware using ball-lock fittings without modification of existing ball-lock hardware.

Apparatus and associated methods relate to a growler cap having a cap body configured to sealingly couple to a growler opening, a pressure pathway through the cap body, and an umbrella valve disposed in fluid communication with the pressure pathway. In an illustrative example, the cap body may include threads configured to engage a threaded growler mouth. In some implementations, the cap body may include a gasket configured to form a pressure-resistant seal against a growler mouth. In some examples, the cap body may be configured to couple to a growler mouth by a clamp, a swing-top mechanism, a bayonet interface, and/or a friction fit. The umbrella valve, for example, may control release of gas from an interior of the growler when pressure exceeds a threshold pressure. In some implementations, the cap body may include a fluid well positioned to receive gas released through the umbrella valve, and a viewing fluid held in the fluid well may form visible bubbles when gas is released. In some examples, gas released through the umbrella valve may be vented to atmosphere without passing through a fluid well. In various implementations, the umbrella valve may be removably received in a valve-receiving aperture and may be replaced with a second umbrella valve associated with a different threshold pressure. In some examples, the growler cap may be used during fermentation, carbonation, storage, transport, and/or serving of a beverage. Various embodiments may allow threshold-based pressure management and passive visual pressure indication to be applied to growler-format vessels during fermentation, carbonation, storage, transport, and/or serving.

Apparatus and associated methods relate to a bottle and cap sealing system having a glass bottle with an internal tapered sealing surface at or near the bottle opening, and a plastic cap configured to engage that internal tapered sealing surface. In an illustrative example, the internal tapered sealing surface may be a conical or frustoconical surface formed on the inside of the glass bottle mouth, so that the plastic cap drops into the mouth and contacts the internal taper in a cone-like seating action. The plastic cap, for example, may include at least one inner sealing fin projecting inwardly from an inner surface of the cap. In some implementations, the inner sealing fin may be an annular fin. In some examples, the plastic cap may include two or more inner sealing fins spaced axially along the inner surface of the cap. The internal tapered sealing surface and the inner sealing fin, for example, may cooperate in a reverse-wedge sealing action such that the sealing fin is deflected or compressed against the internal tapered surface during cap installation. In some implementations, internal pressure acting on the cap and/or the sealing fin may increase the sealing force between the sealing fin and the internal tapered glass surface. In some examples, the pressure-resistant seal may be formed between the plastic sealing fin and the internal glass tapered surface without a separate elastomeric liner. In some implementations, an elastomeric liner or gasket may additionally be disposed between the plastic cap and the glass bottle mouth. The plastic cap, for example, may be retained on the glass bottle by threads, a snap fit, a press fit, a crimp, and/or a bayonet interface. In some implementations, the glass bottle may include a hardened or reinforced base configured to resist warping under internal pressure. In some examples, the plastic cap may further include a pressure pathway, an umbrella valve in fluid communication with the pressure pathway, and a fluid well positioned to receive gas released through the umbrella valve. In various implementations, the bottle and cap sealing system may be used during fermentation, carbonation, storage, transport, and/or serving. Various embodiments may allow a plastic cap—including caps incorporating an umbrella valve and fluid well—to form a pressure-resistant seal on a glass bottle having an internal tapered mouth, so that glass bottle formats may be used in fermentation, carbonation, storage, and transport applications.

Apparatus and associated methods relate to a pressure-responsive bottle system having a plastic bottle configured for hot-fill use, one or more bottle wall regions configured to expand, flex, and/or change shape in response to internal pressure generated during fermentation or carbonation, and a cap or closure coupled to the plastic bottle. In an illustrative example, the plastic bottle may be formed from PET, polyethylene, polypropylene, and/or another thermoplastic configured to withstand hot-fill processing. The one or more bottle wall regions, for example, may include a panel, a rib, a groove, a dome, a base region, a shoulder region, a label panel, a vacuum panel, and/or a sidewall region configured to expand outward under internal pressure. In some implementations, expansion of the one or more bottle wall regions may provide a passive visual indication to a user that internal pressure has increased to or above a selected range. In some examples, expansion of the one or more bottle wall regions may accommodate gas generation and may reduce abrupt pressure rise within the bottle. In some implementations, the cap may include an umbrella valve configured to release gas above a threshold pressure, and the bottle expansion and the umbrella valve threshold may cooperate in managing internal pressure. In some examples, the one or more bottle wall regions may return at least partially toward an unexpanded shape when internal pressure is reduced. In various implementations, the bottle may pass through a sequence of states including: hot-fill filling, cooling and vacuum accommodation, fermentation or carbonation pressure buildup, visible expansion of the one or more wall regions, and optional valve venting if pressure exceeds the threshold. In some implementations, the plastic bottle may include a base region having increased wall thickness or structural reinforcement configured to resist deformation during hot-fill processing and under internal pressure. In some examples, expansion behavior of the one or more bottle wall regions may be tuned by wall thickness, panel depth, rib geometry, bottle material, bottle volume, and/or cap valve threshold. Various embodiments may allow a user to observe fermentation or carbonation progress through visible expansion of the bottle wall, and may reduce abrupt pressure rise within the bottle by accommodating gas generation through controlled wall expansion, particularly when used in combination with a cap incorporating an umbrella valve.

Apparatus and associated methods relate to a modular pressure-control cap system having two or more cap or adapter bodies, each configured to couple to a different vessel or connector interface, and a common umbrella valve receivable by each of the two or more cap or adapter bodies. In an illustrative example, the two or more cap or adapter bodies may include a bottle cap body, a growler cap body, a ball-lock adapter body, a glass-bottle plastic cap body, a hot-fill plastic bottle cap body, a jar cap body, a keg adapter body, and/or a tubing adapter body. Each cap or adapter body, for example, may define a pressure pathway placing the common umbrella valve in fluid communication with a vessel interior or connector pressure region. In some implementations, each of the two or more cap or adapter bodies may include a valve-receiving aperture having a common geometry configured to receive the same umbrella valve. In some examples, the common umbrella valve may be carried by a removable valve cartridge receivable in each of the two or more cap or adapter bodies. Various embodiments may allow a single umbrella valve to be used interchangeably across bottle caps, growler caps, ball-lock adapters, glass-bottle plastic caps, and hot-fill bottle caps, so that pressure-threshold management may be applied consistently across different vessel formats using common components.

Apparatus and associated methods relate to a pressure-control cap or adapter having a body defining a valve-receiving aperture, a first umbrella valve removably receivable in the aperture and associated with a first threshold pressure, and a second umbrella valve removably receivable in the aperture and associated with a second threshold pressure different from the first threshold pressure. In an illustrative example, the first and second umbrella valves may differ in durometer, and the difference in durometer may correspond to the difference in threshold pressure. In some implementations, the first and second umbrella valves may differ in diaphragm thickness, diaphragm geometry, retention-module geometry, and/or stem length. In some examples, the first and second umbrella valves may differ in installed preload resulting from interaction between the valve and the valve-receiving aperture. In some implementations, the first and second umbrella valves may be provided as part of a valve kit including two or more umbrella valves each associated with a different threshold pressure. A user or manufacturer, for example, may select one of the first or second umbrella valves based on beverage type, target carbonation level, vessel pressure rating, fermentation stage, shipping condition, and/or storage condition. In some examples, the valve-receiving aperture may be compatible with the first and second umbrella valves across at least two of: a ball-lock adapter body, a growler cap body, a glass-bottle plastic cap body, and a hot-fill plastic bottle cap body. Various embodiments may allow threshold pressure to be adjusted for different beverages, carbonation targets, vessel pressure ratings, fermentation stages, and/or shipping and storage conditions by replacing one umbrella valve with another, without replacement or modification of the cap or adapter body.

Apparatus and associated methods relate to a pressure-control insert having an insert body configured to be received in a cap, lid, adapter, and/or fitting, an aperture formed in the insert body, and/or an umbrella valve retained in the aperture. In an illustrative example, the insert body may couple to a host cap or adapter body by a snap fit, threads, and/or a press fit. In some implementations, a seal may be formed between the insert body and the host cap or adapter body to direct gas flow through the aperture and umbrella valve. In some examples, the insert body may further define a fluid well positioned to receive gas released through the umbrella valve. In some implementations, the insert body may carry only the umbrella valve without an integrated fluid well, and gas released through the umbrella valve may be vented to an indicator region or to atmosphere defined by the host cap or adapter body. In various implementations, the insert body may be configured to be received in at least two of: a ball-lock adapter body, a growler cap body, a glass-bottle plastic cap body, and a hot-fill plastic bottle cap body. Various embodiments may allow the umbrella valve and its associated aperture geometry to be transferred between different host cap or adapter bodies, and may allow the valve insert to be replaced, cleaned, and/or exchanged independently of the host body. Various embodiments may achieve one or more advantages. For example, some embodiments may include an umbrella valve which may advantageously enable a (predetermined) pressure of fermentation to be reached before a visual indicium is generated.

Some embodiments, for example, may advantageously include a removable tasting module. Such embodiments may, for example, advantageously enable a user to taste test contents in the chamber without contamination.

For example, some embodiments may advantageously include a residue collection module. The collection module may, for example, advantageously enable a user to remove selective residue from the chamber without contamination of the (fermenting) contents.

Some implementations may, for example, advantageously include a secondary seal. The secondary seal may, for example, advantageously allow long term storage of a fermented content in a same bottle used for fermentation. Some implementations may, for example, include an mobile application to advantageously log various environmental parameters of the fermenting content over time against one or more predetermined profile associated with a user-selected flavor.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary Fermentation Monitoring System (FMS) employed in an illustrative use-case scenario.

FIG. 2 is a cross-section view of an exemplary fermentation monitoring cap (FMC).

FIG. 3 is a cross-section view an exemplary FMC with a removable tasting module.

FIG. 4 is a cross-section view an exemplary FMC with a secondary seal.

FIG. 5 shows an exemplary umbrella valve having a pre-loading retention module in a non-deployed mode.

FIG. 6 is a block diagram showing an exemplary fermentation system for taste monitoring.

FIG. 7 is a flow diagram showing an exemplary taste monitoring method using the FMS.

FIG. 8 is a flow diagram showing an exemplary purging method using the FMS.

FIG. 9 depicts an example of a spunding valve adapter in a perspective view.

FIG. 10 is a cross-section view of the spunding valve adapter of FIG. 9.

FIG. 11 depicts an example of an FMC coupled directly to a ball-lock in an in-use configuration on a pressurized vessel.

FIG. 12 depicts an example of a ball-lock valve assembly in a perspective view.

FIG. 13A is a cross-section view of an exemplary thick-walled (e.g., glass) bottle with the FMC installed at the bottle opening. FIG. 13B is a perspective view of the glass bottle of FIG. 13A. FIG. 13C is a side view of the glass bottle of FIG. 13A. FIG. 13D is a top view of the glass bottle of FIG. 13A. FIG. 13E is a bottom view of the glass bottle of FIG. 13A.

FIG. 14 is a cross-section view of an exemplary FMC for use with the glass bottle of FIG. 13A.

FIG. 15 is a perspective view of the FMC of FIG. 14.

FIG. 16 depicts examples of bottles with the FMC installed.

FIG. 17 depicts an example carbonation method showing a sequence of states.

FIG. 18 depicts an example of a pressure-threshold structural transition of a flexible bottle.

FIG. 19 depicts examples of bottle configurations.

FIG. 20 depicts additional examples of bottle configurations.

Appendix A depicts example implementations of one or more embodiments disclosed herein.

Like reference symbols in the various drawings indicate like elements.

### DETAILED DESCRIPTION OF ILLUSTRATIVE EMBODIMENTS

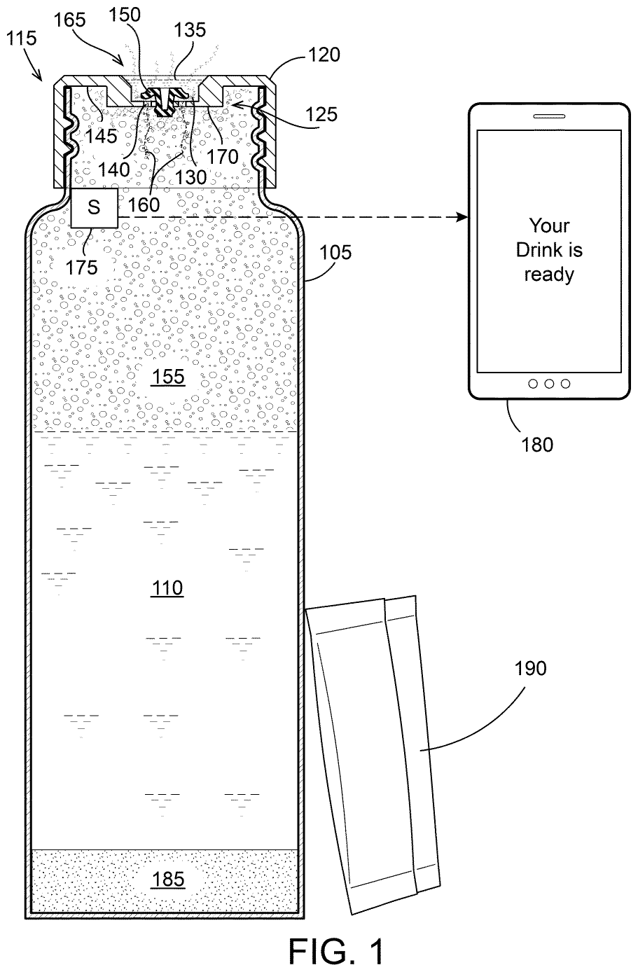

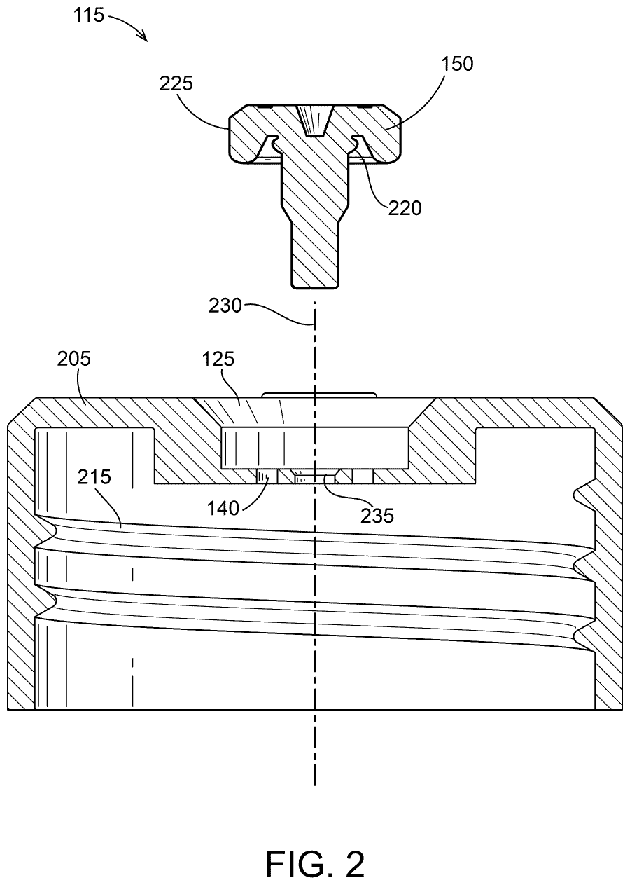

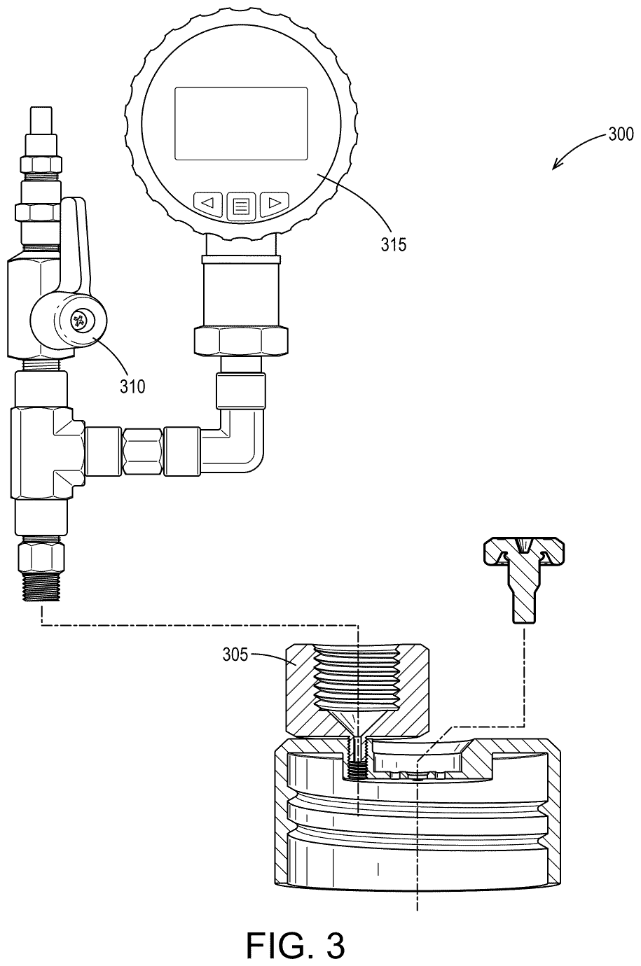

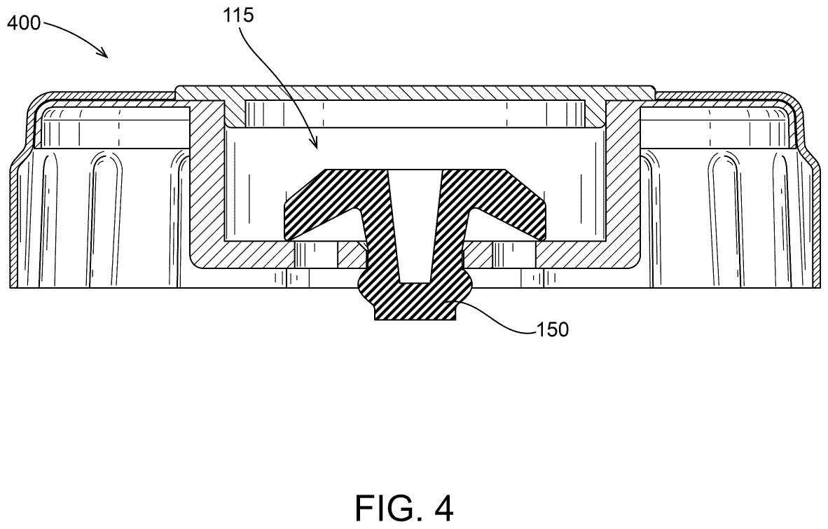

To aid understanding, this document is organized as follows. First, to help introduce discussion of various embodiments, a fermentation monitoring system (FMS) is introduced with reference to FIGS. 1-2. Second, that introduction leads into a description with reference to FIGS. 3-4 of some exemplary embodiments of fermentation monitoring caps (FMC) of the FMS. Finally, the document discusses further embodiments, exemplary applications and aspects relating to FMS.

FIG. 1 depicts an exemplary Fermentation Assist System (FAS 100) employed in an illustrative use-case scenario. For example, a user may use the FAS 100 to brew a drink at home by a fermentation and/or a carbonation process. As an illustrative example, a fermentation-based carbonation process may be performed using the FAS 100. In this example, the FAS 100 is brewing a fermenting beverage 110 contained in a bottle 105. Various liquid is applicable to the FAS 100. For example, the fermenting beverage 110 may be a fermentation fruit juice. For example, the fermentation beverage 110 may be apple juice, pineapple juice, grape juice, other fruit juice, or a mixture thereof. In some examples, the beverage 110 may be kombucha.

In the depicted example, the FAS 100 includes a fermentation monitoring cap (FMC 115). The FMC 115 includes a top surface 120 and a fluid well 125 defined by a well floor 130 at a lower elevation than the top surface 120. In some implementations, the fluid well 125 may contain various kind of viewing fluid 135. For example, the viewing fluid 135 may be water. In some examples, the viewing fluid 135 may be a colored solution. In various implementations, in operation, the viewing fluid 135 may provide a visual indication when the fermentation beverage 110 has met a predetermined condition in a fermentation process.

The FMC 115 includes, in this example, two fluid communication channels 140 at the well floor 130 to an interior surface 145 of the FMC 115. The fluid communication channels 140 are, as shown in FIG. 1, covered by an umbrella valve 150. In some implementations, the umbrella valve 150 may be preloaded with a predetermined tension upon insertion into the FMC 115. For example, the umbrella valve 150 may include a retention bulb that, when relaxed, rest in a plane above a plane of a diaphragm of the umbrella valve 150. Upon insertion to the FMC 115, for example, a tension may be applied to the retention bulb such that a predetermined force may be required to remove the umbrella valve 150 from the FMC 115.

In operation, as depicted in FIG. 1, the FMC 115 is coupled to the bottle 105. For example, the FMC 115 may be releasably threaded to a top opening of the bottle 105. In this example, the bottle 105 and the interior surface 145 formed an enclosed chamber 155. For example, the fermenting beverage 110 may start the fermentation process in the enclosed chamber 155.

In some implementations, the umbrella valve 150 may selectively block air and/or gas flow through the fluid communication channels 140. For example, by selecting a predetermined release pressure of the umbrella valve 150, the fluid communication channels 140 may be set to be unblocked at a predetermined pressure threshold. In some implementations, by way of example and not limitation, the predetermined release pressure of the umbrella valve 150 may be above 25 psi.

During the fermentation process, for example, the fermenting beverage 110 may release gas. For example, the enclosed chamber 155 may experience increasing gas pressure during the fermentation process. In some implementations, when the gas pressure in the enclosed chamber 155 exceeds the predetermined threshold, the gas pressure may force open the fluid communication channels 140 and some escaping gas 160 within the enclosed chamber 155 may escape into the fluid well 125 through the fluid communication channels 140 into the viewing fluid 135. In some examples, the escaping gas 160 may appear as gas bubbles 165 in the viewing fluid 135. In various implementations, the gas bubbles 165 may provide visual indication to the user that the gas pressure has exceeded the predetermined threshold. Accordingly, for example, the user may be visually informed that the fermentation process has met a predetermined condition.

In various implementations, the FAS 100 may advantageously be a safe starter kit for home carbonation/fermentation of juice with automatic pressure release. For example, the FAS 100 may include juice, fermentation catalyst (e.g., inoculant/fermentation culture/probiotic culture), and the FMC 115. In some examples, the FAS 100 may advantageously allow the user to ferment a beverage without constant monitoring to avoid over-pressurization and/or explosion of the fermentation container. In some embodiments, the FMC 115 may advantageously allow bottling of active beverages by avoiding over pressurization.

In various implementations, because the user may select the umbrella valve 150 with a sufficiently high predetermined pressure threshold, the FAS 100 may advantageously allow high pressure fermentation. For example, the fermenting beverage 110 may carbonate and ferment at the same time. Accordingly, the fermenting beverage 110 may, for example, be brewed pre-primed with carbonation during a primary fermentation process. In some examples, in a high-pressure fermentation process, oxygen may be evacuated from the fermenting beverage 110 (e.g., to prevent growth of undesired organisms, such as mold).

In some implementations, the FMC 115 further includes a semi-permeable membrane 170 placed over a vent of the fluid communication channel 140. The semi-permeable membrane 170 may, for example, allow the escaping gas 160 to pass through when the umbrella valve opens, while preventing spillage of the fermenting beverage 110. The semi-permeable membrane 170 may, for example, advantageously enable transportation of the fermenting beverage 110. For example, the fermenting beverage 110 may be transported for distribution to grocery stores without spillage. In some embodiments, the semi-permeable membrane 170 may be a gas-permeable, liquid impermeable membrane that is at least partially transparent. For example, the semi-permeable membrane 170 may allow visualization of the fluid well 125 while preventing spillage of fluid from the fluid well 125.

In some implementations, a semi-permeable membrane (not shown) may be placed over the fluid well 125. The membrane may, by way of example and not limitation, be a membrane such as depicted with respect to the semi-permeable membrane 170. The membrane may, for example, allow the escaping gas 160 to pass through, while preventing the viewing fluid 135 from escaping the fluid well 125 (e.g., spilling out if the container is tipped). Such implementations may, for example, advantageously allow monitoring of the fermentation via the viewing fluid 135 (e.g., visually monitoring for the escaping gas 160), but may prevent the viewing fluid 135 from spilling during transport and/or accidentally (e.g., by getting tipped in a refrigerator). Such implementations may, for example, advantageously enable the cap to be removed without spilling the viewing fluid 135. In some implementations, the membrane may be optically translucent (e.g., such that the viewing fluid 135 is visible through the membrane). In some implementations, the membrane may be substantially optically transparent (e.g., visually clear).

In some implementations, the FMC 115 may include one or more sensors 175. For example, the sensor may be a temperature sensor. In some examples, the sensor may be a pressure sensor. The sensors 175 may, for example, wirelessly communicate with a mobile computing device 180 running an app. In some implementations, the app may log temperature vs time, for example. In some examples, the app may compare the time and temperature to one or more predetermined profiles (e.g., for a user-selected flavor profile). For example, the predetermined profiles may indicate the temperature and pressure profile for the fermenting beverage 110 to have a user-selected taste. In some implementations, the predetermined profiles may be downloadable from the Internet. Upon determining that the fermenting beverage match the predetermined profiles, the app may, for example, generate alerts. For example, the app may generate an audio and/or visual alert to notify the user to check the fermenting beverage 110 when a predetermined change in temperature has happened in a predetermined range of time.

In this example, the FAS 100 further includes a false bottom 185. For example, the false bottom 185 may include a valve to remove dead yeast from the fermenting beverage 110 without contaminating the fermenting contents.

In this example, the FAS 100 further includes a self-dispensing culture package 190. The self-dispensing culture package 190 may, for example, be disposed in the bottle 105 pre-activated initially. In some examples, the package may be activated based on a predetermined operation (e.g., removing a seal, pushing a plunger) from the outside of the bottle 105. Such embodiments may, for example, make dispensing a fermentation culture into the fermenting beverage 110 easier and/or faster. Various such embodiments may advantageously reduce time and/or cost for foodservice and/or supermarket retailers of actively fermenting beverages.

In various implementations, the FMC 115 may include the fluid well 125 and at least one fluid communication channel 140. When the FMC 115 is coupled to the bottle 105 containing the fermenting beverage 110, and the gas pressure within the enclosed chamber 155 exceeds a predetermined threshold, the escaping gas 160 may flow into the fluid well 125 through the fluid communication channels 140 to provide visual indication to the user that the gas pressure has exceeded the predetermined pressure threshold.

FIG. 2 is a cross-section view of an exemplary FMC 115. In this example, the FMC 115 includes a vessel cap 205 and an umbrella valve 150. As shown in FIG. 2, the vessel cap 205 includes threads 215 for coupling with the bottle 105. For example, in operation, the escaping gas 160 may escape from the enclosed chamber to the fluid well 125 via the fluid communication channels 140.

The umbrella valve 150 includes a retention module 220 (e.g., a retention bulb, as depicted) and a substantially circular diaphragm 225. As shown, the umbrella valve 150 may be releasably inserted along an axis 230 into a valve receiving aperture 235. In some examples, an outer circumference of the diaphragm 225 defining a first plane. In some implementations, the retention module 220 may be at rest above the first plane. In this example the retention module 220 is resting within the diaphragm. Accordingly, when the retention bulb is inserted through the valve receiving aperture 235. For example, the outer circumference of the diaphragm 225 may come in contact with a wall of the fluid well 125. For example, the insertion may apply a preloading pressure to the diaphragm based on an elasticity of the retention module 220. In various implementations, the umbrella valve 150 may be 70-45 shore A. In some examples, various hardness may indicate a different predetermined pressure to unblock the fluid communication channels 140. In some implementations, the umbrella valve 150 may be replicable to fit a required predetermined threshold for the fermenting beverage 110. In some implementations the preloaded insertion of the umbrella valve 150 may, for example, advantageously enable application of an umbrella valve to a thin wall.

In some implementations, the preloading pressure may be determined by a thickness of the valve receiving aperture 235. As an illustrative example, the valve receiving aperture 235 may, by way of example and not limitation, be at least 3.5 mm wide. The valve receiving aperture 235 may, by way of example and not limitation, be up to 5 mm wide. In some implementations, a thickness of valve receiving aperture may, for example, be at least 0.9 mm. As an illustrative example, the valve receiving aperture 235 may, by way of example and not limitation, be up to 1.2 mm thick. In some examples, if the thickness is increased (e.g., thereby increasing a separation between the retention module 220 and the umbrella valve 150), the valve receiving aperture 235 may be stretched more. For example, the predetermined pressure to unblock the fluid communication channels 140 may exceed a default value of the umbrella valve 150. For example, the umbrella valve 150 with lower hardness may advantageously be configured to increase an unblock pressure of the fluid communication channels 140. In some implementations, the umbrella valve 150 extending down past a default “pre-loaded” length may increase the predetermined pressure.

FIG. 3 is a cross-section view an exemplary FMC 300 with a removable tasting module 305. For example, the removable tasting module 305 may be a screw port at the top of the FMC 300. In some examples, a user may desire to taste the fermenting beverage 110 to check a progress of the fermentation process. For examples, the user may desire to confirm that no alcoholic content is developing in the fermenting beverage 110. In some examples, the user may remove a portion of the fermenting beverage 110 via the removable tasting module for tasting. In various implementations, the removable tasting module 305 may advantageously allow the user to taste the fermenting beverage 110 without contaminating content of the fermenting beverage 110.

The FMC 300 includes, in this example, a shut off valve 310 (e.g., a Schrader valve) and a gauge 315. For example, the shut off valve 310 and the gauge 315 may be screwed into the top via the removable tasting module 305. In some implementations, the shut off valve 310 may operate the removable tasting module 305. The gauge 315 may, for example, enable measurement of an internal pressure within the bottle 105.

FIG. 4 is a cross-section view an exemplary FMC 115 with a secondary seal 400. In this example, the secondary seal 400 is a crown-cap shaped cover to the FMC 115. For example the secondary seal 400 may fixedly seal the FMC 115 and the bottle 105 with welding solvent, epoxy, and/or other sealing agent. In some implementations, the secondary seal 400 may seal the FMC 115 and the umbrella valve 150 to seal the beverage away from an outside environment. For example, the secondary seal 400 may advantageously allow long term storage of the fermented beverage 110 in the same bottle 105 used for fermentation.

In some embodiments, the secondary seal 400 may be applied once a fermentation process is substantially completed. For example, the secondary seal 400 may be applied after the fermentation process has been started for 7 days, for example.

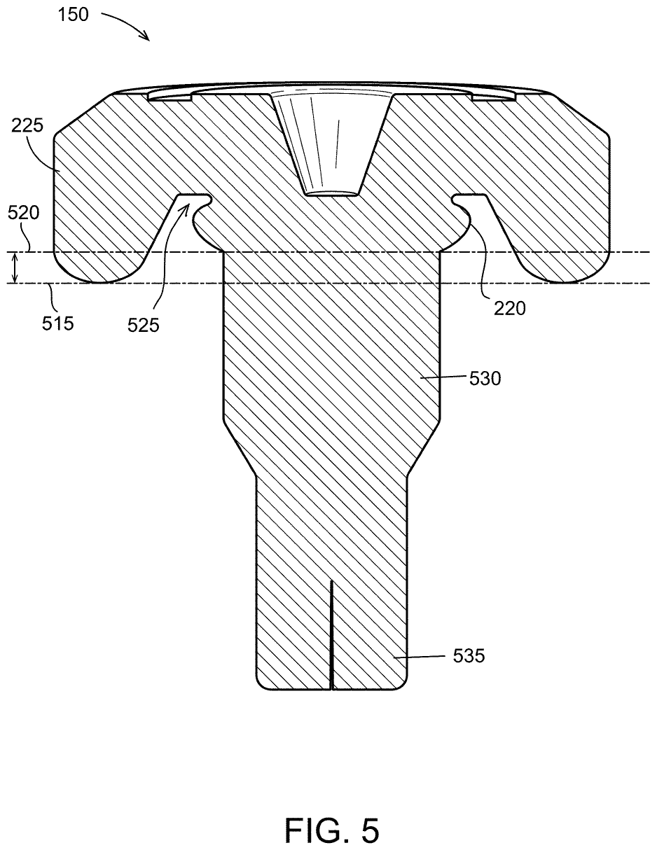

FIG. 5 shows an exemplary umbrella valve 500 having a (pre-loading) retention module 220 in a non-deployed mode. For example, the umbrella valve 500 may be used in the fluid well 125 for assisting fermentation. In this example, the umbrella valve 500 includes a diaphragm 225 at an outer circumference. The outer circumference of the diaphragm 225 rests on a first plane 515. In the non-deployed mode, for example, the retention module 220 is resting at a second plane 520 that, as shown is at a higher elevation than the first plane 515. When the umbrella valve 500 is being deployed, for example, a predetermined pre-loading force may be applied to pull the retention module 220 below the first plane 515.

In some implementations, the umbrella valve 500 may be deployed into the fluid well 125 by applying a predetermined force associated with the hardness of the retention module 220. In some examples, a predetermined tension may be applied to pull the retention module 220 through a wall of the fluid well 125. In some implementations, the predetermined tension may keep the diaphragm 225 substantially sealing with a thin wall of the fluid well 125. In some examples, the the thin wall may be thinner than at least some of the diaphragm of the umbrella valve. The thin wall may, for example, be less than 2 mm. The thin wall may, for example, be less than 1.5 mm. The thin wall may, for example, be less than 1.1 mm. In some examples, the thin wall may be less than 1 mm.

For example, in operations, when the umbrella valve 500 is in the deployed mode operates, the umbrella valve 500 may substantially block the flow communication channels 140. For example, the umbrella valve 500 may maintain a gas pressure within the chamber 155. In some examples, when the gas pressure in the chamber 155 may exceed a predetermined threshold (e.g., 30 psi), the gas pressure may generate a force greater than the predetermined tension of the retention module 220. In some implementations, the umbrella valve 500 may be transiently open. For example, the umbrella valve 500 may temporarily open the flowing communication channels 140 to reduce the gas pressure. In some implementations, the umbrella valve 500 may advantageously maintain the gas pressure within the chamber 155 at the predetermined threshold.

The umbrella valve 150 includes a thin hollow area 525 for contacting the well floor 130. In some implementations, the thin hollow area 525 may be configured to be thin to create a hollow cavity with the well floor 130. For example, a greater area covered by the hollow cavity may be configured such that a higher gas pressure may be required to push up on the umbrella valve 150.

In this example, the umbrella valve 150 includes a valve stem 530. The valve stem 530 includes a tapering tip 535 configured to be tapered thinner into the receiving aperture 235. For example, the tapering tip 535 may allow the umbrella valve 150 to fit through a small valve receiving aperture 235 (e.g., 3.5 mm). In some implementations, the valve stem 530 may, in some portion, thicker than a diameter of the valve receiving aperture 235. In some examples, when the umbrella valve 150 is inserted through the valve receiving aperture 235, a higher friction force may be generated to hold the retention module 220 into place.

For example, a diameter of the receiving aperture 235 may be D1, a diameter of the tapering tip 535 may be D2, and a diameter of the valve stem 530 just above the receiving aperture 235 may be D3. In some implementations, D2<D1<D3. For example, D1 may be 3.5 mm. D2 may, for example, be 3 mm. D3 may, for example, be 4.5 mm. Accordingly, the tapering tip 535 may be inserted into the receiving aperture 235 (e.g., for alignment and/or to enable a user to ‘grip’ the valve to pull it through the hole). The valve stem 530 may then require an interference fit such that a force of friction between the vessel cap 205 and the valve stem 535 and/or the retention module 220 may, for example, advantageously prevent dislodgement of the valve from the vessel cap 205.

In some examples, depending on the content for fermentation (e.g., a carbonated orange juice versus a Kombucha), the predetermined tension required may vary. In some implementations, the umbrella valve 500 with different predetermined tension may be replaceably (e.g., interchangeably, releasably) coupled to the fluid well 125.

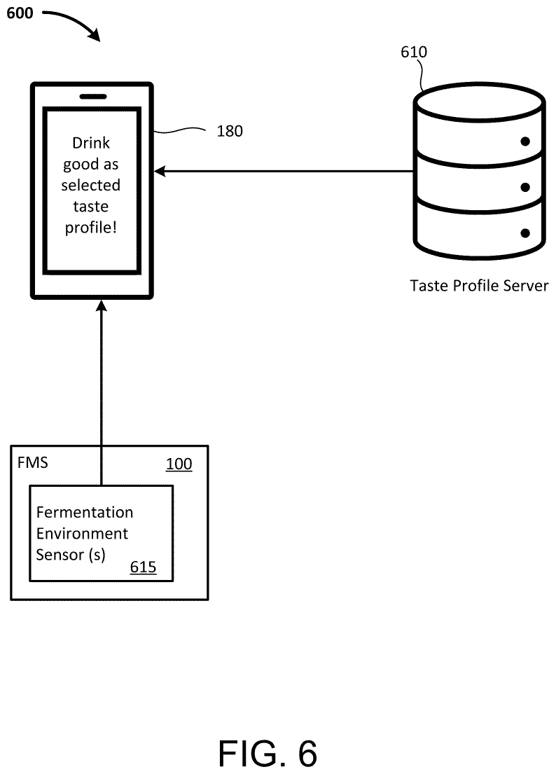

FIG. 6 is a block diagram showing an exemplary fermentation system 600 for taste monitoring. The fermentation system 600 includes the FAS 100, a mobile device 180, a taste profile server 610. As shown in this example, the FAS 100 includes one or more fermentation environment sensors (FES) 615. In some implementations, the FES 615 may be monitoring various environmental parameters associated with the fermenting beverage 110. For example, the FES 615 may include temperature sensors, humidity sensors, pressure sensor, light sensor, gas content sensor, and/or other sensor capable of obtaining environmental reading related to the fermenting beverage 110.

The mobile device 180 may receive the data transmitted from the FES 615. In some implementations, the mobile device 180 may include a mobile app for receiving the data from the FES 615. In this example, the mobile device 180 also receives taste profile data from the taste profile server 610. For example, the taste profile server 610 may include taste profiles associated with a tasting result of a fermenting content (e.g., a fermenting beverage or a fermenting food). In some implementations, the taste profile may include a data structure of environmental parameters at different time points of a specific fermentation content. In some examples, the taste profile may be associated with a guideline (e.g., general guideline, predetermined guidelines) of attributes at different times to get a user-selected taste or texture of the specific fermentation content.

In some implementations, the FES 615 may be setup to monitor environmental attributes of interest related to the taste profiles. In some implementations, a user may select a predetermine taste profile. For example, the mobile app may provide a list of available taste profiles from the taste profile server 610. In some implementations, based on a user-selected taste profile, the mobile app may compare the historical readings from the FES 615 to determine one or more reminders for the user. For example, based on the historical reading of temperature of the fermentation beverage 110, the mobile app may notify the user to check on the fermenting beverage 110 at an appropriated time. In some implementations, the mobile app may notify the user to change a temperature of the fermenting beverage 110 to achieve the user-selected taste profile.

In some implementations, the mobile app may also record the environmental attributes to create new taste profile. For example, after a fermenting beverage is created, the user may choose to upload the historical environmental attributes to the taste profile server 610 as a new taste profile.

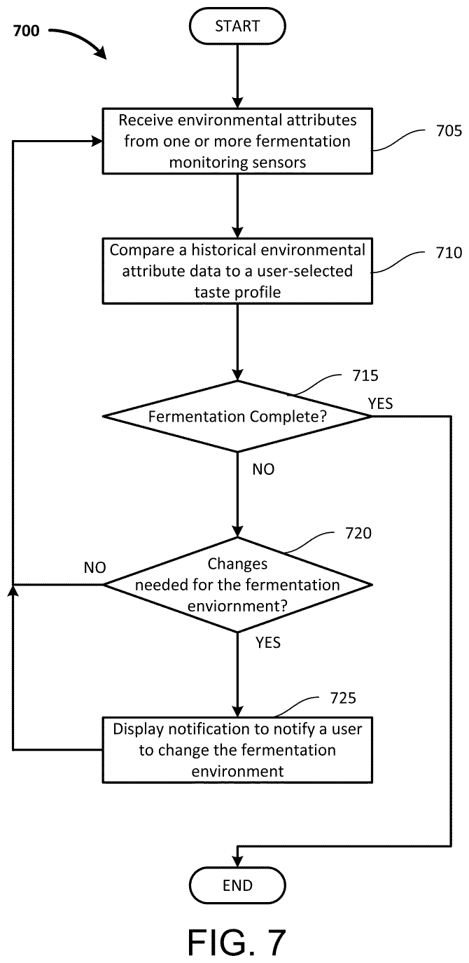

FIG. 7 is a flow diagram showing an exemplary taste monitoring method 700 using the FAS 100. For example, the monitoring method 700 may be executed by the mobile device 180 in the fermentation system 600. In this example, the method 700 starts when environmental attributes are received from one or more fermentation monitoring sensors in a step 705. For example, the mobile device 180 may receive temperature data from the temperature sensor 175.

Upon receiving the environmental attributes, a historical environmental attribute data is compared to a user-selected taste profile 710. For example, the user-selected taste profile may be retrieved from the taste profile server 610 and displayed for user selection. In some examples, the user-selected taste profile may be selected at the beginning of a fermentation process. In some implementations, a user may be allowed to switch the user-selected taste profile during the fermentation process.

In this example, after step 710, it is determined whether a fermentation is completed 715. For example, the mobile app may compare the environmental attributes a set of fermentation completion criterion to determine that the fermentation is completed. If it is it is determined that a fermentation is completed, then the method 700 ends. If it is determined that a fermentation is not completed, then it is determined whether one or more changes are needed for the fermentation environment 720. For example, the mobile app may determine that a lower temperature is needed for achieving the user-selected taste profile. If it is determined that one or more changes are needed for the fermentation environment, in this example, a notification is displayed to notify a user to change the fermentation environment 725. If it is determined that no change is needed for the fermentation environment, the method 700 return to step 705.

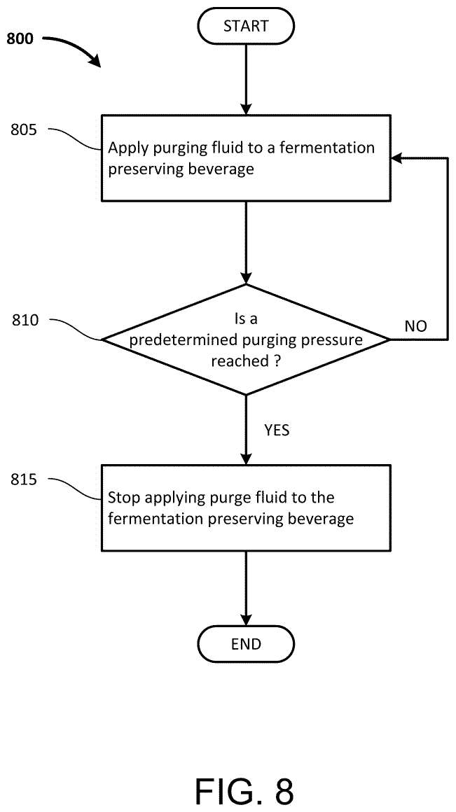

FIG. 8 is a flow diagram showing an exemplary purging method 800 using the FAS 100. In some implementations, the FMC 115 may be used in a preservation mode. For example, the bottle 105 may include a port for applying a purging fluid (e.g., nitrogen gas). In this example, the method 800 starts when a purging fluid is applied to a fermentation preserving beverage 805. The purging fluid may, for example, enter the port and escape through the fluid communication channels 140.

Next, it is determined whether a predetermined purging pressure reached 810. In some implementations, the fluid well 125 may provide a visual indication when a predetermined purging pressure has reached during purging. In some examples, the predetermined purging pressure threshold may, for example, allow a pressure to be reached to drive an existing fluid (e.g., oxygen) out of the enclosed chamber 155. If it is determined that the predetermined purging pressure has not reached, then the step 805 is repeated. If it is determined that the predetermined purging pressure has been reached, then the purge fluid is stopped to apply to the fermentation preserving beverage 815 and the method 800 ends. As soon as the purging fluid ceases being applied, for example, the umbrella valve 150 may close, preserving a positive pressure in the bottle 105. For example, the positive pressure may prevent oxygen from diffusing into the bottle 105.

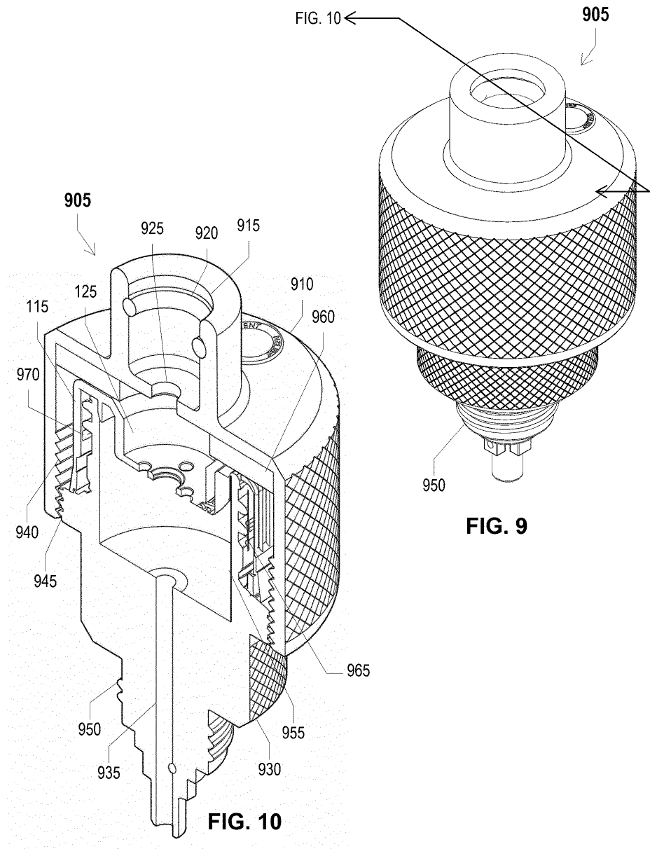

FIG. 9 depicts an example of a spunding valve adapter 905 in a perspective view. In this example, the spunding valve adapter 905 is an adapter configured to receive the fermentation monitoring cap 115 (FMC 115) as a functional pressure-management element of the adapter. In this example, the FMC 115 seals into the spunding valve adapter 905 (e.g., including its umbrella valve 150 and fluid well 125). The FMC 115 with umbrella value 150 may, for example, act as the membrane and valve unit of the assembly. In this example, the spunding valve adapter 905 provides a housing, a pressure pathway, and a container coupling interface. As shown, the spunding valve adapter 905 may place the FMC 115 in fluid communication with the interior of a vessel or fitting to which the spunding valve adapter 905 is coupled.

In this example, the spunding valve adapter 905 includes an upper body 910 and a lower body 930. The upper body 910 and the lower body 930 are coupled together to define the adapter body of the spunding valve adapter 905. In this example, the upper body 910 and the lower body 930 are coupled by an upper body coupler 940 and a lower body coupler 945. In some implementations, such as shown, the upper body coupler 940 and the lower body coupler 945 are cooperating threaded interfaces. In some embodiments the upper body coupler 940 and the lower body coupler 945 may be other cooperating coupler interfaces such as, for example, cooperating snap-fit and/or press-fit interfaces.

As shown, the upper body 910 includes a lumen 915. The lumen 915 extends through the upper body 910 along a central axis of the spunding valve adapter 905. The lumen 915 defines at least a portion of a pressure pathway through the spunding valve adapter 905, as described in greater detail with reference to FIG. 10. The lumen 915 is shown with a seal 920 (e.g., o-ring, as shown). The seal 920 may, for example, fluidly seal a container and/or conduit (e.g., tubing) into the lumen 920. The lumen 915, in this example, communicates with an interior volume of the spunding valve adapter via an aperture 925.

The upper body 910 is configured to receive the FMC 115. In this example, the FMC 115 is received at an upper end of the upper body 910 such that the FMC 115 seals against the upper body 910. In some implementations, the upper body 910 includes threads or other coupling features configured to engage corresponding coupling features of the FMC 115. In some examples, the FMC 115 is received in the upper body 910 by a press fit or snap fit. When the FMC 115 is received in and sealed to the upper body 910, the umbrella valve 150 and the fluid well 125 of the FMC 115 are placed in fluid communication with the lumen 915 and the pressure pathway of the spunding valve adapter 905. In this configuration, the FMC 115 acts as the membrane and valve unit of the spunding valve adapter 905, with the umbrella valve 150 of the FMC 115 controlling gas release through the pressure pathway in response to pressure in the vessel or fitting to which the spunding valve adapter 905 is coupled.

The spunding valve adapter 905 includes a sealing member 960. The sealing member 960 is disposed at or near the interface between the FMC 115 and the upper body 910. In some implementations, the sealing member 960 may be an O-ring or gasket (e.g., as shown) configured to form a pressure-resistant seal between the FMC 115 and the upper body 910 when the FMC 115 is received in the upper body 910. The sealing member 960 may reduce gas leakage at the interface between the FMC 115 and the upper body 910 during pressurized operation. For example, coupling the upper body 910 to the lower body 930 may urge the sealing member 960 into fluid sealing with the FMC 115.

The spunding valve adapter 905 includes a container coupler 950. The container coupler 950 is disposed at a lower end of the lower body 930 in this example. The container coupler 950 may, for example, be configured to couple the spunding valve adapter 905 to a vessel or fitting. In this example, the container coupler 950 includes threads configured to engage a threaded fitting or vessel opening. In some implementations, the container coupler 950 includes a standard soda bottle thread, such as a PCO 1881 thread, configured to engage a corresponding threaded fitting on kegging hardware, a growler, a mini-keg, and/or other vessel and/or connector. In some examples, the container coupler 950 may be configured to engage a ball-lock-compatible fitting or adapter. In some implementations, the container coupler 950 may be configured to engage a gas-line fitting or tubing connector used in homebrewing or commercial beverage production.

FIG. 10 is a cross-section view of the spunding valve adapter 905 of FIG. 9, taken along the central axis of the adapter body. FIG. 10 illustrates the internal pressure pathway connecting the container coupler 950 at the lower end of the spunding valve adapter 905 to the FMC 115 received at the upper end, and shows the sealing relationship between the FMC 115 and the adapter body.

As shown in FIG. 10, the upper body 910 defines the lumen 915 extending through the upper body 910. The lower body 930 defines a lower lumen 935 extending through the lower body 930. When the upper body 910 and the lower body 930 are coupled together by the upper body coupler 940 and the lower body coupler 945, the lumen 915 and the lower lumen 935 together define a continuous pressure pathway extending from the container coupler 950 at the lower end of the spunding valve adapter 905 to the FMC 115 received at the upper end through the umbrella valve (not shown in order to provide better visibility of other components).

In this example, the lower body 930 and the upper body 910 cooperate to define a cavity 955. The cavity 955 is in fluid communication with the lumen 915 and the lower lumen 935.

The FMC 115 is shown received in and sealed between to the upper body 910 at the upper end of the spunding valve adapter 905. In this cross-section view, the fluid well 125 of the FMC 115 is visible at the upper end of the assembly, and the umbrella valve 150 (not shown) of the FMC 115 is positioned to receive gas pressure from the lumen 915 below. The FMC 115 seals against the upper body 910 such that gas traveling upward through the pressure pathway is directed through the fluid communication channels 140 of the FMC 115 and against the umbrella valve 150.

The container coupler 950 is shown in cross-section at the lower end of the lower body 930. The container coupler 950 includes coupling interface(s) configured to engage a vessel or fitting. The sealing member 960 is shown disposed between the FMC 115 and the upper body 910, forming a pressure-resistant seal at that interface.

In operation, the spunding valve adapter 905 may, for example coupled (e.g., to a vessel) such as by engaging the container coupler 950. In some examples, the container coupler 950 engages a threaded port on a growler, mini-keg, and/or gas line fitting. In some implementations, the container coupler 950 engages a PCO 1881 or other standard soda bottle thread on kegging hardware. The FMC 115 may, for example, be coupled to the lower body 930. For example, the FMC 115 may couple (e.g., threadedly, as shown) by FMC coupler 970 to coupler 965 of the lower body 930. In this example, the upper body 910 couples to the lower body 930, with the sealing member 960 forming a pressure-resistant seal between the FMC 115 and the upper body 910.

Gas pressure from the interior of the vessel or fitting travels upward through the lower lumen 935, through the cavity 955, and to the FMC 115. The pressure pathway defined by the lumen 915 and the lower lumen 935 places the interior of the vessel or fitting in fluid communication with the umbrella valve 150 and the fluid well 125 of the FMC 115. In this configuration, the FMC 115 and umbrella valve 150 acts as the membrane and valve unit of the spunding valve adapter 905.

While the gas pressure in the vessel or fitting remains below the predetermined threshold of the umbrella valve 150, the umbrella valve 150 remains closed and the fluid communication channels 140 of the FMC 115 remain blocked. In some embodiments, viewing fluid may be omitted in the fluid well.

When the gas pressure in the vessel or fitting exceeds the predetermined threshold, the umbrella valve 150 opens transiently. Escaping gas 160 travels through the fluid communication channels 140 of the FMC 115 and into the fluid well 125 and through the lumen 915. After the pressure is reduced below the threshold, the umbrella valve 150 returns to its closed position.

In this way, the spunding valve adapter 905 allows the threshold-based pressure management (e.g., with or without passive visual pressure indication of the FMC 115) to be applied to vessels and fittings (e.g., that use threaded or other coupling interfaces compatible with the container coupler 950). The FMC 115 used in the fermentation assist system 100 on a plastic bottle 105 may, for example, also advantageously be usable as the functional element of the spunding valve adapter 905 by sealing the FMC 115 into the upper body 910. In some examples, the umbrella valve 150 of the FMC 115 is replaceable with a second umbrella valve associated with a different threshold pressure, allowing the threshold to be selected for a particular vessel pressure rating, beverage type, carbonation target, fermentation stage, and/or storage condition.

In some implementations, the spunding valve adapter 905 is used during at least one of: purging a vessel with an inert gas, carbonating a beverage, dispensing a beverage, controlling fermentation pressure, and/or managing storage pressure. In some examples, the spunding valve adapter 905 is used in combination with tubing and/or kegging hardware connected to the container coupler 950. Conventional spunding valves used in homebrewing and/or commercial beverage production include mechanical pressure-relief valves that may clog over time and fail in a closed position, stopping pressure relief. Because the umbrella valve 150 of the FMC 115 is a rubber element, a failure mode of the umbrella valve 150 may, for example, advantageously remain open rather than sealing closed. In some examples, this failure mode may reduce the risk of over-pressurization of the vessel compared to a conventional spunding valve that fails closed.

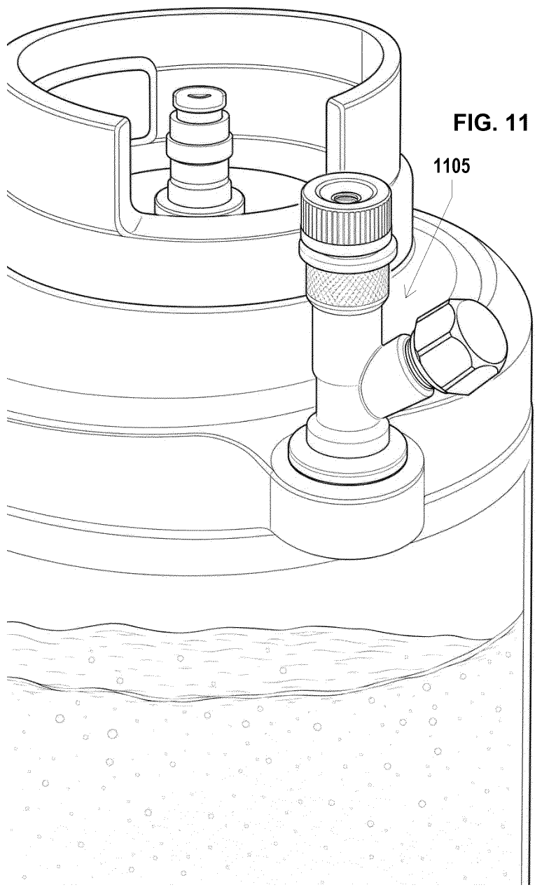

FIG. 11 depicts an example of the FMC 115 coupled directly to a ball-lock 1105 in an in-use configuration on a pressurized vessel. In this example, the FMC 115 is threaded directly onto the ball-lock 1105, with no intermediate adapter body between the FMC 115 and the ball-lock 1105. The ball-lock 1105 is shown coupled to a vessel in this in-use view.

In some examples, the vessel is a growler. In some implementations, the vessel is a mini-keg. In some examples, the vessel is a corny-keg-style vessel. In some implementations, the vessel may be another pressurized vessel having a ball-lock fitting. In this configuration, the FMC 115—including its umbrella valve 150—may be placed in direct fluid communication with the interior of the vessel through the ball-lock 1105, so that the FMC 115 acts as the pressure-management element of the ball-lock valve assembly. In some examples, the FMC 115 may have a fluid well. In some such embodiments, the FMC 115 may advantageously provide visual-indication function.

In operation, gas pressure from the interior of the vessel travels through the ball-lock 1105 to the FMC 115. While the gas pressure remains below the predetermined threshold of the umbrella valve 150, the umbrella valve 150 remains closed. When the gas pressure exceeds the predetermined threshold, the umbrella valve 150 opens transiently, escaping gas 160 passes through the fluid communication channels 140 of the FMC 115 (e.g., into the viewing fluid 135 in the fluid well 125, when so provided, and gas bubbles 165 may form in the viewing fluid 135; the gas bubbles 165 may, for example, provide a passive visual indication to the user that the gas pressure in the vessel has exceeded the predetermined threshold). After the pressure is reduced below the threshold, the umbrella valve 150 returns to its closed position.

In some examples, the ball-lock valve assembly depicted in FIG. 11 may, for example, be used during fermentation of a beverage in the vessel. In some implementations, the assembly may used during carbonation of a beverage. In some examples, the assembly may be used during storage and/or transport of a pressurized beverage. In some implementations, the assembly may, for example, be used during purging of the vessel with an inert gas such as nitrogen or carbon dioxide. In some examples, the assembly may, for example, be used during dispensing of a beverage from the vessel.

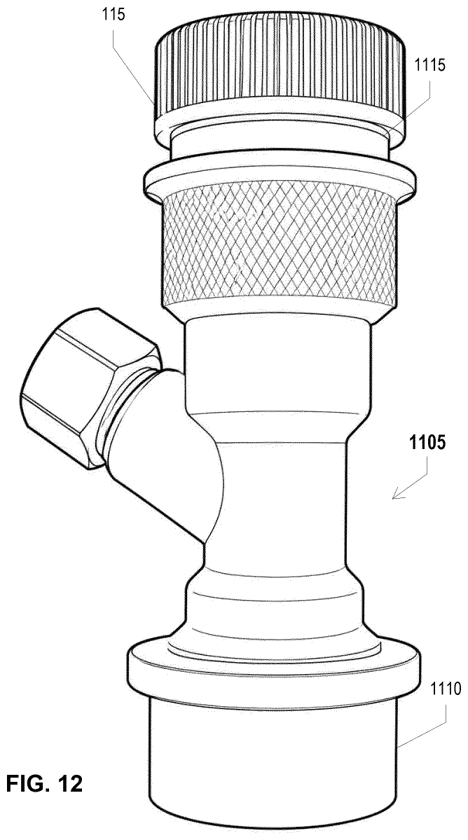

FIG. 12 depicts an example of the ball-lock valve assembly in a perspective view, showing the FMC 115 coupled directly to the ball-lock 1105 via the valve coupler 1115. FIG. 12 illustrates the structural relationship between the FMC 115, the valve coupler 1115, the ball-lock 1105, and the container coupler 1110.

The ball-lock 1105 may, for example, include a ball-lock fitting of the type used on growlers, mini-kegs, corny-keg-style vessels, and/or other ball-lock-equipped vessels and hardware. The ball-lock 1105 includes a valve coupler 1115. The valve coupler 1115 may, for example, be a coupling feature of the ball-lock 1105 configured to receive and engage the FMC 115 directly. In this example, the valve coupler 1115 includes threads configured to engage corresponding threads of the FMC 115, such that the FMC 115 is threaded directly onto the ball-lock 1105 at the valve coupler 1115, even without an intermediate adapter body.

The ball-lock 1105 includes a container coupler 1110. The container coupler 1110 may be configured to couple the ball-lock 1105 to a vessel. In some implementations, the container coupler 1110 may be configured to engage a ball-lock post on a growler, mini-keg, and/or corny-keg-style vessel. In some examples, the container coupler 1110 may include a ball-lock-compatible interface of the gas-post or liquid-post type.

The FMC 115 is shown coupled to the valve coupler 1115 of the ball-lock 1105 in this perspective view. In this configuration, the fluid well 125 of the FMC 115 is visible at the upper end of the assembly, and the umbrella valve 150 of the FMC 115 is positioned to receive gas pressure from the ball-lock 1105 through the valve coupler 1115. The FMC 115 in this example may, for example, be a smaller version of the FMC 115 used in the fermentation assist system 100, configured to couple to the valve coupler 1115 of the ball-lock 1105 while retaining the umbrella valve 150. As shown, the FMC 115 may also include the fluid well 125. The fluid well may be provided in some examples with viewing fluid 135. The FMC 115 may include fluid communication channels 140 of the FMC 115.

In some implementations, the valve coupler 1115 may include a PCO 1881 or other standard soda bottle thread, and the FMC 115 may, for example, include corresponding threads configured to engage the valve coupler 1115. In some examples, the valve coupler 1115 and the FMC 115 may be coupled, for example, by a snap fit and/or press fit. In some implementations, the ball-lock 1105 may, for example, be a Kegland-compatible or other commercially available ball-lock fitting having a threaded port configured to receive a soda-bottle-thread cap. The FMC 115 is directly receivable on the ball-lock 1105 without modification of the ball-lock 1105.

In some examples, the umbrella valve 150 of the FMC 115 may, for example, be replaceable with a second umbrella valve associated with a different threshold pressure. Such examples may, for example, advantageously allow the threshold pressure of the ball-lock valve assembly to be selected for a particular vessel pressure rating, beverage type, carbonation target, fermentation stage, and/or storage condition. In some implementations, a user selects a threshold pressure by selecting an umbrella valve 150 of a corresponding durometer, diaphragm thickness, diaphragm geometry, retention-module geometry, and/or installed preload. In some examples, a kit of two or more umbrella valve 150 units, each associated with a different threshold pressure, is provided for use with the ball-lock 1105, so that a user selects the appropriate umbrella valve 150 for a given application by replacing one umbrella valve 150 in the FMC 115 with another.

In some implementations, the ball-lock valve assembly of FIGS. 11 and 12 is used in place of a conventional ball-lock valve, such as in homebrewing or commercial beverage production. Conventional spunding valves may include mechanical pressure-relief elements that may clog and fail in a closed position. Because the umbrella valve 150 of the FMC 115 is a rubber element, a failure mode of the umbrella valve 150 may advantageously remain open rather than sealing closed, which may advantageously reduce the risk of over-pressurization of the vessel. In some examples, the threshold pressure of the ball-lock valve assembly may be selected by replacing the FMC 115 or umbrella valve 150 with a second FMC 115 or umbrella valve 150 associated with a different threshold pressure (e.g., rather than by adjusting a mechanical knob or setting on the assembly). In some implementations, this advantageously allows a user to change the threshold pressure of the assembly by exchanging one FMC 115 for another.

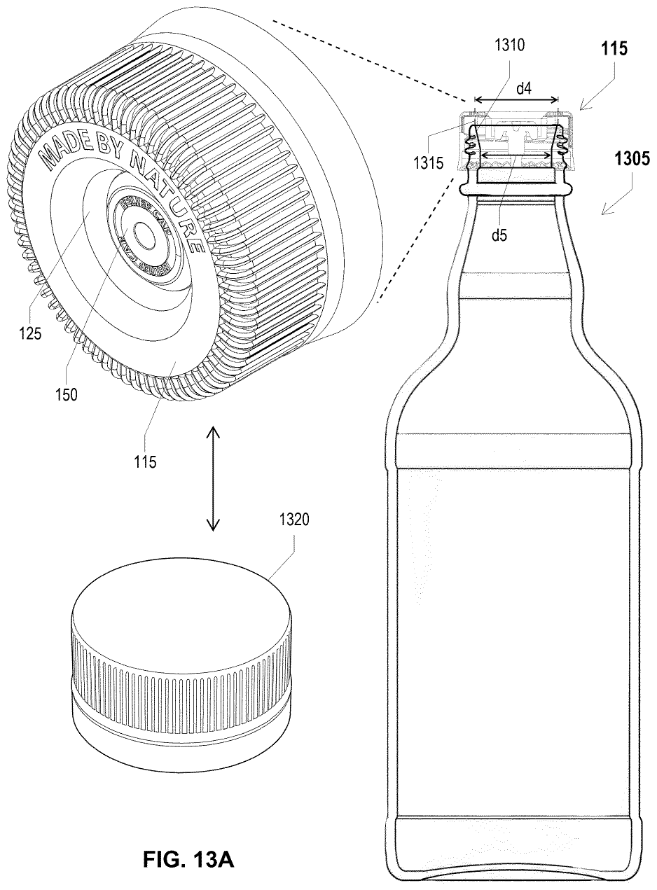

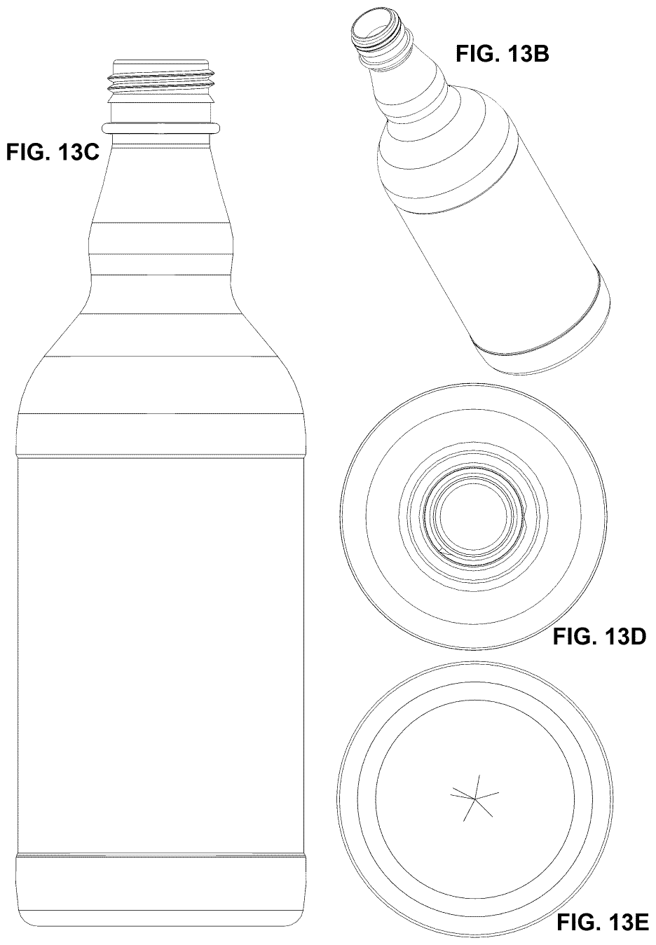

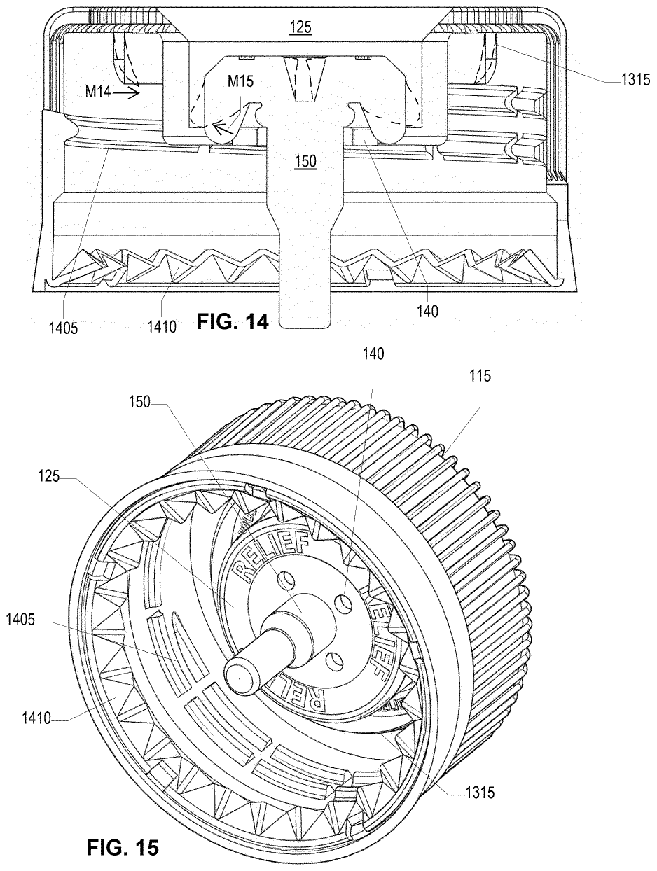

FIGS. 13A-13E depict an example of a glass bottle 1305. FIG. 13A depicts the glass bottle 1305 coupled to the FMC 115. FIGS. 13B-13E depict the glass bottle 1305 alone. FIGS. 14 and 15 depict the FMC 115 in cross-section and perspective views, respectively, in the context of use with the glass bottle 1305.

In this example, the glass bottle 1305 includes an inner contour 1310 at or near the bottle opening. The inner contour 1310 is a tapered surface formed on the inside of the glass bottle mouth. The FMC 115 includes a sealing member 1315. The sealing member 1315 is disposed on an inner surface of the FMC 115 at a position corresponding to the inner contour 1310 when the FMC 115 is installed on the glass bottle 1305. The inner contour 1310 and the sealing member 1315 cooperate in a reverse-wedge sealing action during installation of the FMC 115 on the glass bottle 1305. The sealing interface between the sealing member 1315 and the inner contour 1310 is a plastic-on-glass contact in this example.

In some examples, the pressure-resistant seal is formed between the sealing member 1315 and the inner contour 1310 without a separate elastomeric liner between the FMC 115 and the glass bottle 1305. In some implementations, an elastomeric liner or gasket is additionally disposed between the FMC 115 and the glass bottle mouth. In some examples, the FMC 115 used with the glass bottle 1305 includes the umbrella valve 150 and the fluid well 125 described with reference to FIGS. 1 and 2. The inner contour 1310 of the glass bottle 1305 is one example of a structural modification to a glass bottle mouth that may allow a plastic cap (e.g., including a plastic cap incorporating an umbrella valve and fluid well) to form a pressure-resistant seal on the glass bottle 1305 without a separate elastomeric liner.

FIG. 13A is a cross-section view of the glass bottle 1305 with the FMC 115 installed at the bottle opening. FIG. 13A illustrates the inner contour 1310, the sealing member 1315, and the sealing relationship between the FMC 115 and the glass bottle 1305.

The glass bottle 1305 includes the inner contour 1310. The inner contour 1310 is formed on the inside of the glass bottle mouth at or near the bottle opening. In this example, the inner contour 1310 is a conical or frustoconical tapered surface. The inner contour 1310 defines a first diameter d4 at a first axial position and a second diameter d5 at a second axial position along the central axis of the bottle opening. In this example, d4 is greater than d5. The inner contour 1310 narrows inward from the first axial position toward the second axial position. In some implementations, the first axial position is at or near the top of the bottle opening and the second axial position is at a lower elevation within the bottle mouth.

In some examples, the inner diameter varies relative to the position along the central longitudinal axis, as shown. The inner diameter may vary monotonically, for example. As shown, in this example the diameter is linearly varying.

The inner contour 1310 is a feature of the glass bottle 1305 itself. In some examples, the inner contour 1310 may be formed during manufacture of the glass bottle 1305 by molding, pressing, and/or blowing the glass to define the tapered inner surface at the bottle mouth. In some implementations, the inner contour 1310 may be a frustoconical surface. In some examples, the inner contour 1310 includes a curved, chamfered, and/or compound taper geometry. In some implementations, the inner contour 1310 is formed below the threaded finish of the glass bottle 1305, within the interior of the bottle mouth. A standard glass bottle mouth may, for example, be substantially flat or cylindrical on the inside. The inner contour 1310 may, for example, represent a departure from that standard geometry. Although a glass bottle is discussed in this example, other materials may be used. For example, relatively thick-walled bottles may be polymeric. Thick-walled bottles may, for example, be metallic (e.g., multi-wall construction). Thick-walled bottles may, for example, be wooden. Thick-walled bottles may, for example, be ceramic. In some examples, thick walls may be at least 1.5 mm. Thick walls may, for example, be 2 mm or more. Thick walls may meet or exceed 2.5 mm in some embodiments. In some examples, thick walls may be 3 mm or more.

The FMC 115 is shown installed on the glass bottle 1305 in FIG. 13A. The FMC 115 includes the sealing member 1315. The sealing member 1315 is disposed on the inner surface of the FMC 115. In some implementations, the sealing member 1315 is, as shown in this example, an annular sealing fin projecting inwardly from the inner surface of the FMC 115. In some examples, the FMC 115 includes two or more sealing members 1315 spaced axially along the inner surface of the FMC 115.

As shown in FIG. 13A, when the FMC 115 is installed on the glass bottle 1305, the sealing member 1315 contacts the inner contour 1310. The sealing member 1315 is deflected (e.g., motion M14 as shown in FIG. 14) and/or otherwise compressed against the inner contour 1310 in a reverse-wedge sealing action during installation. The sealing member 1315 may, for example, be a plastic element. The inner contour 1310 may, for example, be a glass surface in this example. The sealing interface may, for example, be a plastic-on-glass contact. In some implementations, the sealing member 1315 deforms against the inner contour 1310 during installation. The resulting compressive contact is one example of a pressure-resistant seal between the FMC 115 and the glass bottle 1305.

In some examples, when internal pressure in the glass bottle 1305 acts on the FMC 115 during pressurized operation, the pressure may urge the FMC 115 outward along the central axis of the bottle opening. This outward urging may increase the compressive contact force between the sealing member 1315 and the inner contour 1310. In some implementations, this pressure-assisted sealing action may reduce gas leakage at the interface between the FMC 115 and the glass bottle 1305 during pressurized operation.

The fluid well 125 of the FMC 115 is visible at the upper end of the assembly in FIG. 13A. The umbrella valve 150 of the FMC 115 is positioned to receive gas pressure from the interior of the glass bottle 1305 through the fluid communication channels 140 of the FMC 115, as described in greater detail with reference to FIG. 14.

FIG. 13B is a perspective view of the glass bottle 1305 alone. FIG. 13B illustrates the overall form of the glass bottle 1305 and the bottle opening at the upper end.

In this example, the glass bottle 1305 includes a threaded finish at the bottle opening. The inner contour 1310 is formed on the inside of the bottle mouth at or near the bottle opening. The inner contour 1310 is not visible in this perspective view.

In some examples, the glass bottle 1305 may be a glass bottle of a type used for fermented beverages, carbonated beverages, kombucha, juice, and/or other beverages. In some implementations, the glass bottle 1305 includes a hardened and/or reinforced base region. In some examples, the base region may reduce warping of the glass bottle 1305 under internal pressure during pressurized operation. In some implementations, the glass bottle 1305 is a hot-fill-compatible glass bottle. In some examples, the glass bottle 1305 includes a threaded finish of a standard format, such as a PCO 1881 thread (e.g., modified to include the inner contour 1310 at the bottle mouth).

FIG. 13C is a side view of the glass bottle 1305. The opposite side of the glass bottle 1305 is a mirror image of the side shown in FIG. 13C. FIG. 13C illustrates the overall profile of the glass bottle 1305, including the bottle body, shoulder, neck, and threaded finish at the bottle opening. FIG. 13D is a top view of the glass bottle 1305, looking downward along the central axis of the bottle opening. In this view, the threaded finish of the glass bottle 1305 is visible at the perimeter of the bottle opening. The inner contour 1310 is visible as the tapered inner surface of the bottle mouth inward of the threaded finish. FIG. 13E is a bottom view of the glass bottle 1305, looking upward along the central axis of the bottle. In some implementations, the base region of the glass bottle 1305 includes increased wall thickness or structural reinforcement. In some examples, the increased wall thickness or structural reinforcement of the base region may reduce deformation of the glass bottle 1305 during pressurized operation.

FIG. 14 is a cross-section view of the FMC 115 such as shown in FIG. 13A. FIG. 14 illustrates the coupling features 1405, the retention features 1410, the sealing member 1315, the fluid communication channels 140, the umbrella valve 150, and the fluid well 125 of the FMC 115. FIG. 14 also depicts the seal engagement motion M14 and the valve deflection motion M15.

The FMC 115 includes coupling features 1405. The coupling features 1405 are disposed on an outer surface of the FMC 115. In some implementations, such as shown, the coupling features 1405 are threads. In some examples, the coupling features 1405 are a PCO 1881 or other standard thread. In some implementations, the coupling features 1405 are snap-fit, press-fit, bayonet, and/or crimp features. The coupling features 1405 are at a position corresponding to a threaded finish and/or other coupling feature of the glass bottle 1305. In some examples, engagement of the coupling features 1405 with the glass bottle 1305 may retain the FMC 115 on the glass bottle 1305 during pressurized operation.

The FMC 115 includes retention features 1410. The retention features 1410 are structural features of the inner surface of the FMC 115. The retention features 1410 may, for example, resist radial and/or axial displacement (e.g., after first installation).

The FMC 115 includes the sealing member 1315. The sealing member 1315 is disposed on the inner surface of the FMC 115. In some implementations, the sealing member 1315 is an annular inner sealing fin projecting inwardly from the inner surface of the FMC 115. In some examples, the sealing member 1315 may include a flexible sealing lip and/or rib. In some implementations, the FMC 115 may include two or more sealing members 1315 (e.g., spaced axially along the inner surface of the FMC 115).

The seal engagement motion M14 is depicted in FIG. 14. The seal engagement motion M14 is the axial motion of the FMC 115 relative to the glass bottle 1305 in the direction that advances the sealing member 1315 into contact with the inner contour 1310 during installation. In some implementations, the seal engagement motion M14 corresponds to a rotational screwing motion associated with engagement of the coupling features 1405 with the threaded finish of the glass bottle 1305. In some examples, the seal engagement motion M14 may include a linear pressing or snapping motion.

In some implementations, the FMC 115 used with the glass bottle 1305 may omit the fluid well 125 and/or the umbrella valve 150. For example, the FMC 115 may be a solid cap, such as cap 1320. The cap 1320 may, as shown, have a continuous outer surface without a fluid well 125, without fluid communication channels 140, and/or without an umbrella valve 150. In some such implementations, the solid cap may include the sealing member 1315 configured to engage the inner contour 1310 of the glass bottle 1305 in the reverse-wedge sealing action described herein. A sealing member 1315 of the solid cap may, for example, contact and be deflected against the inner contour 1310 during installation in the same manner as described with reference to FIGS. 13A and 14, forming a pressure-resistant seal between the solid cap and the glass bottle 1305 (e.g., at a plastic-on-glass interface in a configuration in which the cap 1320 includes plastic). In some examples, internal pressure acting on the solid cap may urge the solid cap outward along the central axis of the bottle opening, increasing the compressive contact force between the sealing member 1315 and the inner contour 1310. The solid cap may, for example, include the coupling features 1405 (e.g., threads, snap-fit features, press-fit features, bayonet features, and/or crimp features) configured to engage corresponding coupling features of the glass bottle 1305 to retain the solid cap on the glass bottle 1305 during pressurized operation.

In some implementations, the solid cap may be used on the glass bottle 1305 for storage, transport, and/or serving of a beverage that does not rely on active pressure relief during the sealed period. For example, a beverage that has fermented and has been chilled to arrest further gas production may be sealed with the solid cap for long-term storage. In some examples, the solid cap may be used on the glass bottle 1305 for beverages that are not actively fermenting, such as, by way of example and not limitation, pasteurized beverages, still beverages, and/or beverages that have been force-carbonated to a stable equilibrium pressure within the pressure rating of the glass bottle 1305. In some implementations, the solid cap may be used as a replacement closure after the FMC 115 with the umbrella valve 150 has been used during an active fermentation or carbonation phase. For example, a user may install the FMC 115 with the umbrella valve 150 and the fluid well 125 during fermentation. The user may (e.g., subsequently) replace the FMC 115 with the solid cap, such as for long-term sealed storage and/or for serving.

In some examples, the solid cap and the FMC 115 with the umbrella valve 150 may share a common outer geometry and common coupling features 1405, such that both caps are receivable on the same glass bottle 1305 without modification of the glass bottle 1305. In some implementations, the solid cap and the FMC 115 with the umbrella valve 150 may share a common inner geometry (e.g., including the sealing member 1315, such as at the same axial position). For example, both caps may be configured to engage the inner contour 1310 of the glass bottle 1305 in a (e.g., same) reverse-wedge sealing action. In some examples, the solid cap may include the retention features 1410 on the inner surface of the solid cap. The solid cap may, for example, be formed from the same thermoplastic material as the FMC 115, such as polyethylene, polypropylene, and/or PET. In some implementations, the solid cap may be a one-piece molded plastic cap with the sealing member 1315 integrally molded with the body of the solid cap.

In some implementations, the glass bottle 1305 with the inner contour 1310 may be used with a cap that omits the umbrella valve 150 but retains the fluid well 125. For example, such a cap may include the fluid well 125 and the fluid communication channels 140 without the umbrella valve 150, such that gas may pass freely through the fluid communication channels 140 into the fluid well 125 without a threshold pressure. In some examples, the glass bottle 1305 with the inner contour 1310 may be used with a cap that omits the fluid well 125 but retains the umbrella valve 150. For example, such a cap may include the umbrella valve 150 and a vent outlet to atmosphere without a fluid well 125, such that gas released through the umbrella valve 150 is vented to atmosphere without passing through a viewing fluid 135. In various implementations, the inner contour 1310 of the glass bottle 1305 may cooperate with the sealing member 1315 of any of the foregoing cap configurations—including the solid cap, the cap with the fluid well 125 and the umbrella valve 150, the cap with the fluid well 125 without the umbrella valve 150, and the cap with the umbrella valve 150 without the fluid well 125—to form a pressure-resistant seal at the plastic-on-glass interface.

The valve deflection motion M15 is depicted in FIG. 14. The valve deflection motion M15 is the deflection of the umbrella valve 150 as it contacts the bottom of the fluid well 125. Pressure release may, for example, also cause a further deflection motion M15.

The FMC 115 includes the fluid communication channels 140. The fluid communication channels 140 extend through the FMC 115 from the interior of the glass bottle 1305 to the umbrella valve 150. The umbrella valve 150 is positioned at the fluid communication channels 140. The fluid well 125 is positioned above the umbrella valve 150.

In operation, when the FMC 115 is installed on the glass bottle 1305 by the seal engagement motion M14, the sealing member 1315 is deflected against the inner contour 1310 by the valve deflection motion M15. Gas pressure from the interior of the glass bottle 1305 travels through the fluid communication channels 140 to the umbrella valve 150. While the gas pressure remains below the predetermined threshold of the umbrella valve 150, the umbrella valve 150 remains closed. When the gas pressure exceeds the predetermined threshold, the umbrella valve 150 opens transiently (motion M15). Escaping gas 160 passes through the fluid communication channels 140 into the fluid well 125. Gas bubbles 165 form in the viewing fluid 135 (e.g., when viewing fluid is provided in the fluid well 125). The gas bubbles 165 are one example of a passive visual indication to a user that the gas pressure in the glass bottle 1305 has exceeded the predetermined threshold. After the pressure is reduced below the threshold, the umbrella valve 150 returns to its closed position.

In some examples (e.g., which may be implemented in various embodiments/applications shown herein), the umbrella valve may be configured such that the gas escape correlates to gas production (e.g., in fermenting). Such embodiments may, for example, advantageously enable a substantially continuous escape of gas. A substantially continuous escape of gas may, for example, advantageously enable a substantially continuous visual indication. By way of example and not limitation, the thickened umbrella valve walls, the rounded tip of the umbrella wall, the partial depth cavity, and/or the durometer of the valve may advantageously be selected to allow just enough deflection (motion M15) to permit gas to escape while structurally resisting an ‘over-release’ in which the gas pressure is released as a bolus.

In some examples, the FMC 115 used with the glass bottle 1305 is the same FMC 115 used in the fermentation assist system 100 on a plastic bottle 105. In some implementations, the umbrella valve 150 of the FMC 115 is replaceable with a second umbrella valve associated with a different threshold pressure. In some examples, the glass bottle 1305 is used in premium beverage applications where a glass bottle format is selected over a plastic bottle format.

FIG. 15 is a perspective view of the FMC 115. The underneath of the fluid well 125 is visible in this perspective view. The umbrella valve 150 is positioned within the FMC 115 below the fluid well 125 and is not visible in this perspective view. The coupling features 1405 are visible on the outer surface of the FMC 115. The sealing member 1315 is disposed on the inner surface of the FMC 115

In some implementations, the FMC 115 depicted in FIG. 15 is a one-piece plastic cap. In some examples, the FMC 115 is formed from a thermoplastic material such as polyethylene, polypropylene, and/or PET. In some implementations, the sealing member 1315 is integrally molded with the body of the FMC 115. In some examples, the sealing member 1315 is a separately formed element assembled into the FMC 115. In some implementations, the FMC 115 is a disposable cap. In some examples, the FMC 115 is a reusable cap.

In some implementations, the FMC 115 depicted in FIG. 15 is usable on the glass bottle 1305 and on a plastic bottle 105 having a compatible threaded finish. In some examples, the coupling features 1405 of the FMC 115 are a PCO 1881 or other standard soda bottle thread. The glass bottle 1305 includes a corresponding threaded finish in some examples. In some such examples, the FMC 115 is directly receivable on the glass bottle 1305 without modification of the FMC 115.

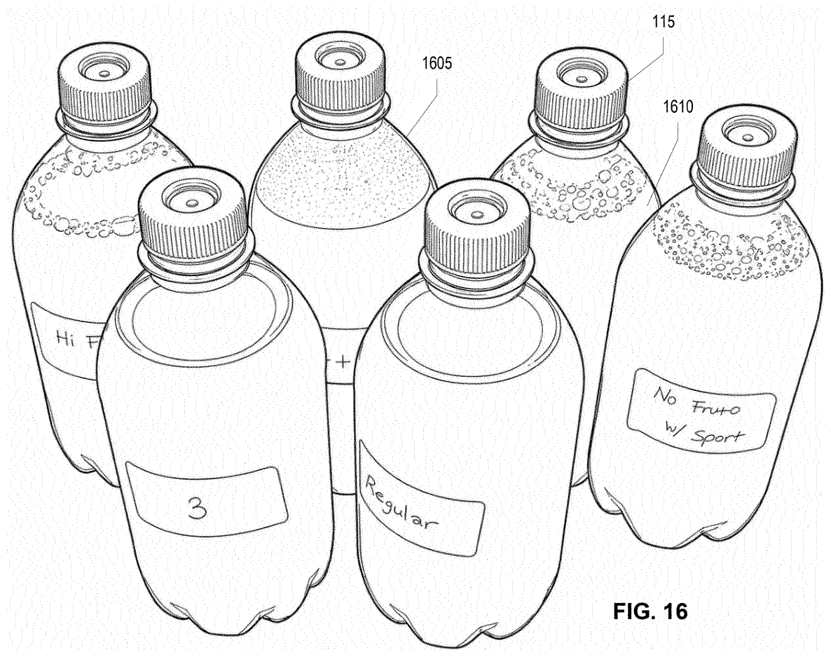

FIG. 16 depicts examples of plastic bottles 1605 with the FMC 115 installed. FIG. 16 illustrates the FMC 115 in the context of use on plastic bottles 1605 of different sizes and configurations. In this example, the plastic bottles 1605 each include a threaded finish at the bottle opening. The FMC 115 is coupled to the threaded finish of each plastic bottle 1605 in this example.

In some examples, the plastic bottles 1605 may be standard soda-bottle-format bottles having a PCO 1881 or other standard threaded finish. In some implementations, the plastic bottles 1605 are formed from PET, polyethylene, polypropylene, and/or another thermoplastic material. In some examples, the plastic bottles 1605 may be of different volumes, such as 500 mL, 1 L, or 2 L formats.

The FMC 115 is shown installed on each of the plastic bottles 1605 in FIG. 16. The umbrella valve 150 and the fluid well 125 of the FMC 115 are visible. In some examples, a viewing fluid 135 may be held in the fluid well 125. In some implementations, the fluid 1610 in the plastic bottles 1605 is a beverage undergoing fermentation or carbonation.

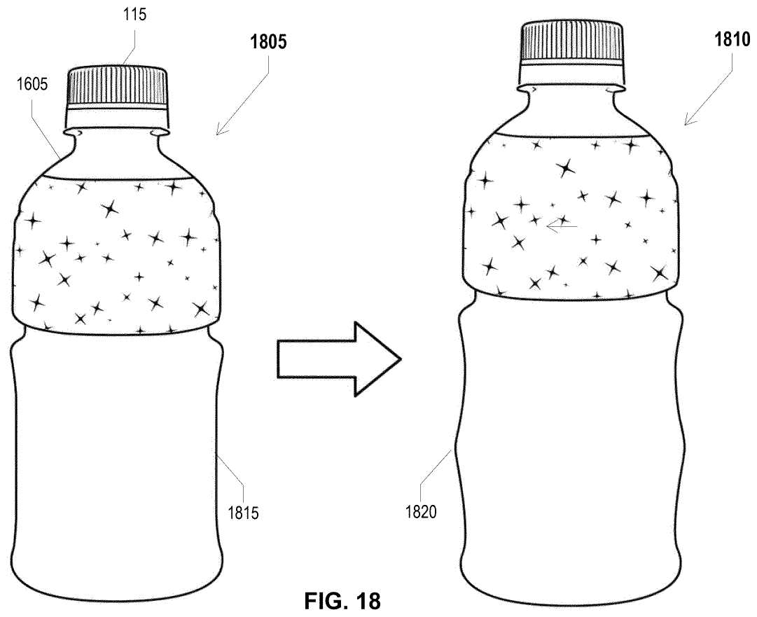

In some examples, the plastic bottles 1605 include one or more bottle wall regions configured to expand, flex, and/or change shape in response to internal pressure generated during fermentation or carbonation, as described in greater detail with reference to FIG. 18. In some implementations, the plastic bottles 1605 are hot-fill-compatible bottles. In some examples, the plastic bottles 1605 include structural features configured to respond to internal pressure changes during fermentation or carbonation.

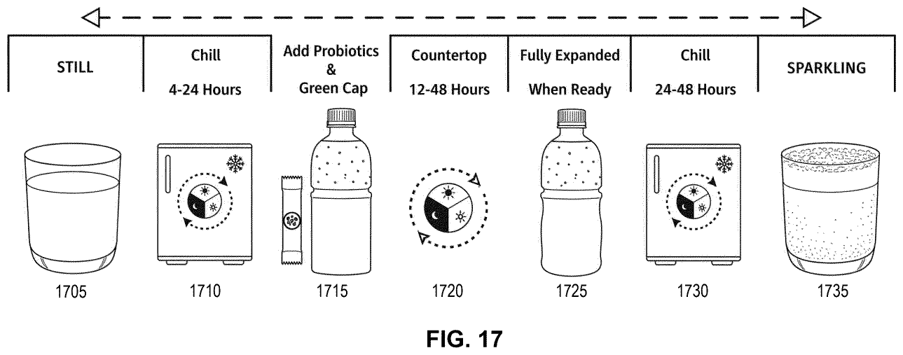

FIG. 17 depicts an example carbonation method. In this example, the carbonation method includes a sequence of states 1705-1735. FIG. 17 illustrates the sequence of states a beverage and bottle assembly may pass through during a fermentation-based carbonation process using the FMC 115 and a plastic bottle 1605. State 1705 depicts an example starting state. In this example, state 1705 includes a glass with fluid. In some examples, the fluid may be a juice, a juice-and-water mixture, a kombucha base, and/or another fermentable beverage. In some implementations, the fluid is a pre-mixed beverage ready for inoculation with a fermentation culture.

State 1710 depicts a chilling step. In this example, the beverage may be chilled prior to addition of a fermentation culture. In some implementations, chilling the beverage before inoculation may reduce the activity of undesired microorganisms in the beverage prior to introduction of the selected fermentation culture.

State 1715 depicts addition of a fermentation culture to the beverage. In this example, a probiotic and/or other fermentation culture is added to the chilled beverage. In some implementations, the fermentation culture includes a probiotic starter culture. In some examples, the fermentation culture includes a yeast, a bacterial culture, a kombucha SCOBY, and/or another inoculant. In some examples, the fermentation culture is added from the culture package 190 such as described with reference to FIG. 1.

State 1720 depicts an incubation step. In this example, the inoculated beverage is held at a selected temperature for a selected period of time. In some implementations, the incubation period is in a range of approximately 4 to 24 hours. In some examples, the incubation period and temperature are selected based on a target flavor profile and/or carbonation level.

State 1725 depicts a fermentation step. In this example, the inoculated beverage undergoes fermentation in the plastic bottle 1605 with the FMC 115 installed. In some implementations, fermentation generates carbon dioxide gas within the enclosed chamber 155 defined by the plastic bottle 1605 and the FMC 115. In some examples, the gas pressure in the enclosed chamber 155 increases during the fermentation step. In some implementations, the one or more bottle wall regions of the plastic bottle 1605 may expand, flex, and/or otherwise change shape in response to the increasing internal pressure during the fermentation step, such as described in greater detail with reference to FIG. 18. In some examples, when the gas pressure in the enclosed chamber 155 exceeds the predetermined threshold of the umbrella valve 150, the umbrella valve 150 opens transiently and escaping gas 160 passes into the fluid well 125, forming gas bubbles 165 in the viewing fluid 135.

State 1730 depicts a second chilling step. In this example, after fermentation has reached a desired level, the plastic bottle 1605 with the FMC 115 installed is chilled. In some implementations, chilling the fermented beverage may reduce the activity of the fermentation culture and/or may slow or arrest further gas production. In some examples, chilling may also increase the solubility of carbon dioxide in the beverage, which may increase the carbonation level of the beverage.

State 1735 depicts a ready-to-serve state. In this example, the fermented and chilled beverage is ready for consumption. In some implementations, the FMC 115 is removed from the plastic bottle 1605 and replaced with a standard cap or serving closure before serving. In some examples, the beverage is served directly from the plastic bottle 1605.

FIG. 18 depicts an example of a pressure-threshold structural transition of the plastic bottle 1605. FIG. 18 illustrates a sub-threshold configuration 1805 and a supra-threshold configuration 1810 of the plastic bottle 1605, and identifies an expansion region 1815 and an expanded rib 1820 of the plastic bottle 1605.

In this example, the plastic bottle 1605 includes one or more bottle wall regions configured to expand, flex, and/or change shape in response to internal pressure generated during fermentation or carbonation. FIG. 18 illustrates the plastic bottle 1605 in two states: the sub-threshold configuration 1805, in which the internal pressure is below a bottle expansion threshold, and the supra-threshold configuration 1810, in which the internal pressure is at or above the bottle expansion threshold.

The bottle expansion threshold is a pressure level at which the one or more bottle wall regions of the plastic bottle 1605 begin to expand, flex, and/or change shape. In some examples, the bottle expansion threshold is lower than the predetermined threshold of the umbrella valve 150 of the FMC 115. In some such examples, the plastic bottle 1605 may begin to visibly expand before the umbrella valve 150 opens. In some implementations, this sequence may allow a user to observe a first passive visual indication—the visible expansion of the bottle wall—at a lower pressure, and a second passive visual indication—gas bubbles 165 in the viewing fluid 135—at a higher pressure when the umbrella valve 150 opens.

In the sub-threshold configuration 1805, the internal pressure in the enclosed chamber 155 is below the bottle expansion threshold. In this configuration, the one or more bottle wall regions of the plastic bottle 1605 are in a baseline or unexpanded state. In some examples, the baseline state corresponds to the shape of the plastic bottle 1605 as filled, sealed, and cooled (e.g., after hot-fill processing). In some examples, hot-fill processing may not be used. In some implementations, the baseline state includes inwardly deflected and/or collapsed vacuum panels (e.g., resulting from cooling contraction of the bottle contents after hot-fill processing). In some examples, the baseline state includes a substantially flat and/or inwardly curved label panel and/or sidewall region.

In the supra-threshold configuration 1810, the internal pressure in the enclosed chamber 155 is at or above the bottle expansion threshold. In this configuration, the one or more bottle wall regions of the plastic bottle 1605 have expanded, flexed, and/or otherwise changed shape in response to the internal pressure. The expansion region 1815 is visible in the supra-threshold configuration 1810 as an outwardly expanded region of the bottle wall. The expanded rib 1820 is visible in the supra-threshold configuration 1810 as an outwardly expanded rib feature of the bottle wall.

In some examples, the transition from the sub-threshold configuration 1805 to the supra-threshold configuration 1810 is visible to a user observing the plastic bottle 1605 from the outside. The visible change in the shape of the plastic bottle 1605 may, for example, be one example of a passive visual indication to a user that the internal pressure has reached or exceeded the bottle expansion threshold. In some implementations, the visible expansion of the bottle wall may allow a user to observe the pressure state of the plastic bottle 1605 without opening the bottle or using a separate pressure gauge.

The expansion region 1815 is a region of the bottle wall of the plastic bottle 1605 configured to expand outward in response to internal pressure. In this example, the expansion region 1815 may include a label panel and/or sidewall region of the plastic bottle 1605. In some implementations, the expansion region 1815 may include a vacuum panel of a plastic bottle. In some examples, the expansion region 1815 may include a dome, a rib, a groove, a shoulder region, a base region, and/or another bottle wall feature configured to respond to internal pressure.

The expansion region 1815 includes structural features that facilitate a controlled outward expansion at the bottle expansion threshold. In some examples, the structural features of the expansion region 1815 may include reduced wall thickness relative to adjacent bottle wall regions. In some implementations, the structural features may include panel geometry, rib geometry, groove geometry, and/or dome geometry configured to respond to internal pressure at the bottle expansion threshold. In some examples, the expansion behavior of the expansion region 1815 may be adjusted by varying wall thickness, panel depth, rib geometry, bottle material, bottle volume, and/or cap valve threshold.

In some implementations, the expansion region 1815 may include a hot-fill vacuum panel. Hot-fill vacuum panels may be structural features of hot-fill type plastic bottles configured to accommodate the volume contraction of bottle contents during cooling after hot-fill processing. In some examples, the inward deflection of the vacuum panel during cooling and the outward expansion of the vacuum panel during fermentation or carbonation pressure buildup may together provide a range of visible bottle-wall states corresponding to different internal pressure states. In some implementations, the vacuum panel geometry of a hot-fill plastic bottle is one example of a bottle wall structure that may be adapted to serve as the expansion region 1815 in a fermentation or carbonation application.

The expanded rib 1820 is a rib feature of the plastic bottle 1605 that is in an outwardly expanded state in the supra-threshold configuration 1810. In some examples, the expanded rib 1820 is a circumferential rib of the plastic bottle 1605. In some implementations, the expanded rib 1820 is a vertical rib, a diagonal rib, and/or a rib of another orientation. In some examples, the expanded rib 1820 is a rib feature of the expansion region 1815. In some implementations, the expanded rib 1820 is a separate rib feature of the bottle wall adjacent to the expansion region 1815.

In some examples, the outward expansion of the expanded rib 1820 in the supra-threshold configuration 1810 increases the internal volume of the plastic bottle 1605 relative to the sub-threshold configuration 1805. In some implementations, the increase in internal volume may accommodate gas generated during fermentation or carbonation. In some examples, the accommodation of gas volume by the outward expansion of the bottle wall may reduce the rate of pressure rise within the plastic bottle 1605 during fermentation or carbonation. In some implementations, the reduction in the rate of pressure rise may reduce the frequency of umbrella valve 150 opening events during fermentation or carbonation.

In some implementations, the plastic bottle 1605 and the FMC 115 cooperate in managing internal pressure during fermentation or carbonation. In this example, the bottle expansion threshold is lower than the predetermined threshold of the umbrella valve 150. In some examples, as internal pressure increases during fermentation or carbonation, the plastic bottle 1605 first transitions from the sub-threshold configuration 1805 to the supra-threshold configuration 1810 at the bottle expansion threshold. The visible expansion of the bottle wall provides a first passive visual indication to a user that internal pressure has increased to the bottle expansion threshold.

If internal pressure continues to increase above the bottle expansion threshold and reaches the predetermined threshold of the umbrella valve 150, the umbrella valve 150 opens transiently. Escaping gas 160 passes through the fluid communication channels 140 into the fluid well 125. Gas bubbles 165 form in the viewing fluid 135. The gas bubbles 165 provide a second passive visual indication to the user that the internal pressure has exceeded the predetermined threshold of the umbrella valve 150.

In some implementations, the combination of the bottle expansion threshold and the umbrella valve threshold may provide two distinct passive visual indications at two different pressure levels. In some examples, the first passive visual indication—bottle wall expansion—may indicate to a user that fermentation or carbonation is progressing. In some examples, the second passive visual indication—gas bubbles in the viewing fluid—may indicate to a user that the internal pressure has reached the umbrella valve threshold. In some implementations, this two-stage indication may allow a user to monitor the progress of fermentation or carbonation without opening the bottle or using a separate pressure gauge.

In some examples, the outward expansion of the bottle wall in the supra-threshold configuration 1810 may reduce the likelihood of abrupt pressure rise within the plastic bottle 1605 by accommodating gas volume. In some implementations, this may reduce the frequency or duration of umbrella valve 150 opening events. In some examples, the combination of bottle wall expansion and umbrella valve venting may together manage internal pressure across a range of fermentation and carbonation conditions.

In some implementations, after the internal pressure is reduced—for example, by umbrella valve venting, by chilling the beverage, and/or by opening the bottle—the one or more bottle wall regions of the plastic bottle 1605 may return at least partially toward the sub-threshold configuration 1805. In some examples, the return of the bottle wall toward the sub-threshold configuration 1805 may provide a further passive visual indication to a user that internal pressure has been reduced.

In some examples, the structural transition of the plastic bottle 1605 from the sub-threshold configuration 1805 to the supra-threshold configuration 1810 may accommodate internal pressure that would otherwise act on the bottle wall without a controlled expansion path. In some implementations, the controlled outward expansion of the expansion region 1815 at the bottle expansion threshold may reduce stress concentrations in other regions of the bottle wall during pressurized operation. In some examples, the bottle expansion threshold being lower than the predetermined threshold of the umbrella valve 150 may allow the bottle wall to begin accommodating internal pressure before the umbrella valve 150 opens. In some implementations, this sequence may reduce the internal pressure at which the umbrella valve 150 is called upon to vent, which may in turn reduce the frequency of venting events.

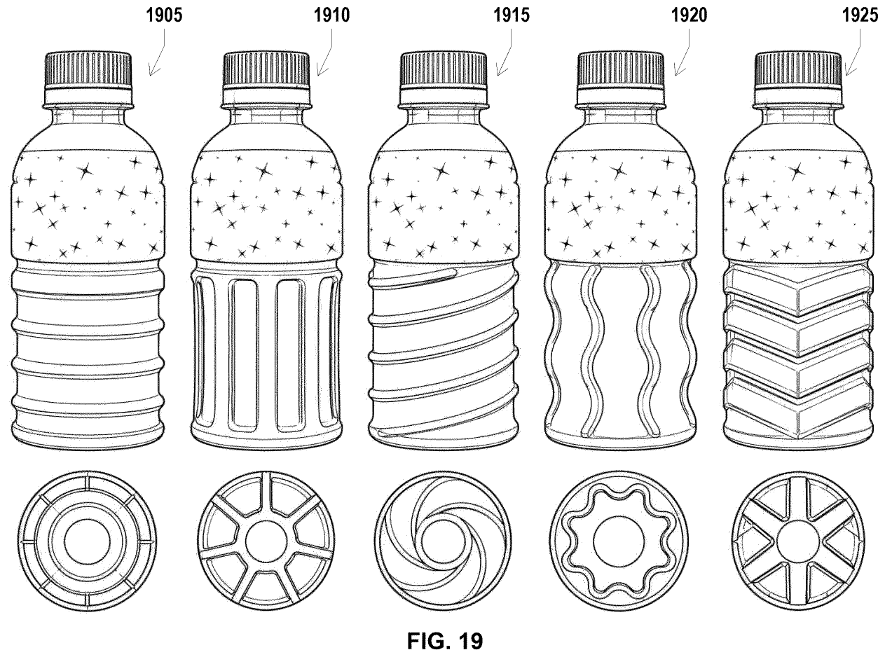

FIG. 19 depicts examples of bottle configurations 1905, 1910, 1915, 1920, and 1925. In this example, each configuration depicts a plastic bottle 1605 in a different shape or geometry. In some examples, the configurations 1905-1925 represent different bottle form factors which may, for example, be compatible with the pressure-responsive bottle system described with reference to FIG. 18. In some implementations, each of the configurations 1905-1925 includes one or more bottle wall regions configured to expand, flex, and/or otherwise change shape in response to internal pressure, such as described with reference to FIG. 18. In some examples, the FMC 115 is coupled to each of the configurations 1905-1925.

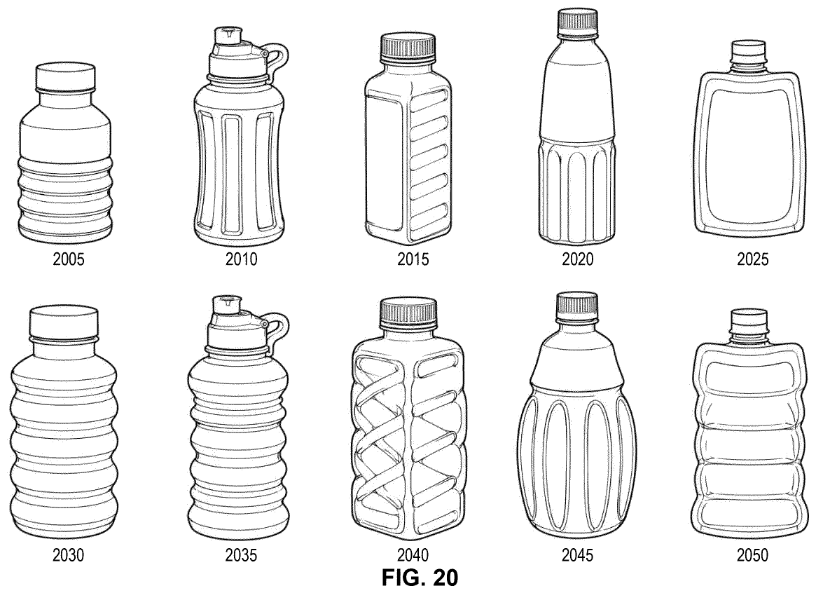

FIG. 20 depicts additional examples of bottle configurations 2005, 2010, 2015, 2020, 2025, 2030, 2035, 2040, 2045, and 2050. In this example, each configuration depicts a container (e.g., plastic bottle 1605) in a different shape/geometry. In some examples, the configurations 2005-2050 represent bottle form factors compatible with the pressure-responsive bottle system such as described with reference to FIG. 18. In some implementations, each of the configurations 2005-2050 includes one or more bottle wall regions configured to expand, flex, and/or otherwise change shape in response to internal pressure, such as described with reference to FIG. 18. In some examples, the FMC 115 is coupled to each of the configurations 2005-2050.

Appendix A depicts example implementation(s) of one or more embodiments disclosed herein. These example implementation(s) are provided by way of example and not limitation. Appendix A is incorporated herein in its entirety.

Although various embodiments have been described with reference to the figures, other embodiments are possible.

In some implementations, other types of stopping valves may be used in place of the umbrella valve 150. For example, the FMC 115 may include a stopping valve having a preloaded tension such that the escaping gas 160 may flow through the stopping valve when the gas pressure in the enclosed chamber 155 has reached the predetermined threshold.

Although an exemplary system has been described with reference to FIGS. 1-4, other implementations may be deployed in other industrial, scientific, medical, commercial, and/or residential applications.

The FAS 100, the FMC 115, the umbrella valve 150, the spunding valve adapter 905, the ball-lock assembly of FIG. 11, the glass bottle 1305, the pressure-responsive plastic bottle 1605, and/or the modular cap platform described herein may be used in various applications and/or markets. The following sections describe example implementations across a range of use cases. Each section introduces an example context, describes particular implementations, and identifies example characteristics of that use case that may correspond to one or more advantages of the described system.

A home user fermenting juice, kombucha, water kefir, ginger beer, and/or another fermentable beverage in a sealed plastic or glass bottle faces a practical challenge: without opening the bottle, there is ordinarily no way to know whether the internal pressure has reached a meaningful level. Opening the bottle to check disrupts the fermentation environment. Leaving the bottle sealed without any pressure indication creates a risk of unexpected pressure release when the bottle is eventually opened.

In some examples, a home user places a fermentable juice in a plastic bottle 1605. The user adds a fermentation culture from the self-dispensing culture package 190. The user installs the FMC 115 on the bottle 1605. The FMC 115 includes the umbrella valve 150 and the fluid well 125 with viewing fluid 135. The user places the sealed bottle in a warm location and observes the fluid well 125 periodically.

The umbrella valve 150 is selected with a threshold pressure corresponding to a target carbonation level for the beverage. In some examples, the user selects an umbrella valve 150 of a particular durometer from a kit of two or more umbrella valves, each associated with a different threshold pressure. The kit may include valves corresponding to, for example, a lightly carbonated beverage, a moderately carbonated beverage, and a highly carbonated beverage.

When the gas pressure in the bottle exceeds the threshold, the umbrella valve 150 opens transiently and gas bubbles 165 form in the viewing fluid 135. The gas bubbles 165 are observable by the user from outside the bottle without opening the bottle. The user observes the gas bubbles 165 as a passive indication that the fermentation process has reached the threshold pressure.

In another example, the home user uses the pressure-responsive plastic bottle 1605 described with reference to FIG. 18. The bottle wall expands visibly when internal pressure reaches the bottle expansion threshold. The visible expansion of the bottle wall is a first passive indication to the user that fermentation is progressing. When the internal pressure subsequently reaches the umbrella valve threshold, gas bubbles 165 in the viewing fluid 135 provide a second passive indication.

In some examples, the home user installs the sensors 175 in the FMC 115 and uses the mobile computing device 180 to log temperature versus time and compare the fermentation environment to a user-selected taste profile. The mobile computing device 180 may generate an alert when the fermentation environment matches the taste profile.

The passive visual indication of the gas bubbles 165 and the bottle wall expansion may allow a home user to monitor fermentation progress without opening the bottle, without using a separate pressure gauge, and without specialized brewing knowledge. The selectable umbrella valve threshold may allow the same FMC 115 to be used across a range of beverages and carbonation targets. The self-dispensing culture package 190 may reduce the time and steps involved in inoculating the beverage. The mobile application may allow a home user to track fermentation conditions against a selected flavor profile without manual logging.

A foodservice operator or small commercial beverage producer fermenting or carbonating beverages in batches faces challenges related to consistency, safety, and efficiency. Batch-to-batch variation in fermentation conditions can result in under-carbonated or over-carbonated beverages. Over-pressurization of sealed containers during fermentation or storage creates a safety risk and a product loss risk. Monitoring internal pressure across multiple containers simultaneously without opening each container is time-consuming.

In some examples, a foodservice operator fills a quantity of plastic bottles 1605 with a fermentable juice and installs the FMC 115 on each bottle. The FMC 115 on each bottle includes the same umbrella valve 150, selected for a threshold pressure corresponding to the target carbonation level of the batch. The operator places the bottles in a fermentation environment and observes the fluid wells 125 of multiple bottles simultaneously. Gas bubbles 165 in the viewing fluid 135 of any bottle indicate that the internal pressure in that bottle has exceeded the threshold.

In another example, the operator uses the glass bottle 1305 with the FMC 115 for a premium product line. The glass bottle 1305 includes the inner contour 1310 at the bottle mouth. The FMC 115 includes the sealing member 1315 configured to engage the inner contour 1310 in a reverse-wedge sealing action. The FMC 115 also includes the umbrella valve 150 and the fluid well 125. The glass bottle format may be selected for premium product presentation. The same FMC 115 used on plastic bottles 1605 is usable on the glass bottle 1305 in this example.

In another example, the operator uses the spunding valve adapter 905 on a mini-keg or growler during a batch fermentation process. The FMC 115 is received in the spunding valve adapter 905. The umbrella valve 150 of the FMC 115 acts as the pressure-management element of the adapter. The operator selects the umbrella valve 150 threshold based on the vessel pressure rating and the target carbonation level of the batch.

In some examples, the operator uses the removable tasting module 305 of the FMC 300 to draw a sample from the fermenting batch for quality control without opening the bottle or contaminating the contents. The shut-off valve 310 and gauge 315 of the FMC 300 may allow the operator to measure the internal pressure of the batch at a selected point in the fermentation process.

The passive visual indication of the gas bubbles 165 across multiple bottles may allow an operator to monitor a batch of fermenting bottles simultaneously without opening each bottle. The common FMC 115 across plastic and glass bottle formats may reduce the number of different cap types an operator stocks. The spunding valve adapter 905 may allow the same umbrella valve platform to be applied to growler and mini-keg formats without separate pressure-management hardware. The removable tasting module 305 may allow quality control sampling without contaminating the batch.

A craft brewer or homebrewer fermenting beer, cider, mead, and/or another brewed beverage in a growler, mini-keg, and/or corny-keg-style vessel may use ball-lock fittings for gas and liquid transfer. Existing ball-lock pressure-management options include mechanical spunding valves that may clog or fail in a closed position. A passive, threshold-based pressure-management and visual-indication system compatible with existing ball-lock hardware and the same umbrella valve platform used on bottles may reduce separate, incompatible pressure-management hardware across different vessel formats.

In some examples, a homebrewer uses the ball-lock assembly of FIG. 11 on a growler during a secondary fermentation process. The FMC 115 is threaded directly onto the ball-lock 1105 on the growler. The umbrella valve 150 of the FMC 115 is selected for a threshold pressure corresponding to the target carbonation level of the beer. When the gas pressure in the growler exceeds the threshold, the umbrella valve 150 opens transiently and gas bubbles 165 form in the viewing fluid 135 of the FMC 115. The brewer observes the gas bubbles 165 as a passive indication that the fermentation pressure has reached the threshold.

In some examples, the brewer uses the spunding valve adapter 905 on a corny-keg-style vessel during a forced carbonation process. The FMC 115 is received in the spunding valve adapter 905. The umbrella valve 150 is selected for a threshold pressure corresponding to the target carbonation level of the beer. The spunding valve adapter 905 is coupled to the vessel through the container coupler 950. The brewer applies carbon dioxide to the vessel and observes the fluid well 125 of the FMC 115. When gas bubbles 165 appear in the viewing fluid 135, the brewer is passively informed that the internal pressure has reached the threshold.

In some examples, the brewer uses the purging method 800 of FIG. 8 to purge a vessel with nitrogen or carbon dioxide before filling. The FMC 115 is installed on the vessel. The brewer applies the purging gas to the vessel through a port. When gas bubbles 165 appear in the viewing fluid 135, the brewer is passively informed that the purging pressure has been reached. The brewer stops applying the purging gas. The umbrella valve 150 closes and retains a positive pressure in the vessel.

In some examples, the brewer selects the umbrella valve 150 threshold by replacing one umbrella valve with another from a kit. Different valves in the kit correspond to different threshold pressures for different beer styles, carbonation levels, and/or vessel pressure ratings.

The direct compatibility of the FMC 115 with existing ball-lock fittings may allow a homebrewer to add threshold-based pressure management and passive visual indication to existing ball-lock-equipped vessels without modifying those vessels. The rubber umbrella valve 150 failure mode—remaining open rather than sealing closed—may reduce the risk of over-pressurization of the vessel compared to a mechanical spunding valve that fails closed. The selectable threshold may allow the same FMC 115 and umbrella valve platform to be used across different beer styles, carbonation targets, and vessel pressure ratings.

A beverage producer distributing actively fermenting beverages—such as kombucha, water kefir, and/or naturally carbonated juice—through retail channels faces a challenge: internal pressure in sealed bottles continues to build during distribution and storage. Bottles may be over-pressurized by the time they reach the consumer. A passive, threshold-based pressure-relief system integrated into the bottle cap may reduce the risk of over-pressurization during distribution and storage without the consumer or retailer having to take any action.

In some examples, a beverage producer fills plastic bottles 1605 with an actively fermenting kombucha and installs the FMC 115 on each bottle. The FMC 115 includes the umbrella valve 150 selected for a threshold pressure corresponding to the maximum safe internal pressure for the bottle format. The semi-permeable membrane 170 is installed over the fluid well 125 to reduce the risk of spillage of the viewing fluid 135 during transport and distribution.

During distribution and storage, the umbrella valve 150 releases gas transiently when the internal pressure exceeds the threshold, then closes. The internal pressure is maintained at or below the threshold throughout distribution. When the consumer receives the bottle, the internal pressure is within the intended range.

In another example, the producer uses the pressure-responsive plastic bottle 1605 of FIG. 18. The bottle wall expands visibly when internal pressure reaches the bottle expansion threshold. A consumer observing the bottle on a retail shelf may observe the expanded bottle wall as a passive indication that the beverage is actively fermenting and carbonated.

In some examples, the producer uses the secondary seal 400 of FIG. 4 to seal the FMC 115 on the bottle after fermentation is substantially complete. The secondary seal 400 may reduce the risk of the FMC 115 being removed or tampered with during distribution.

In some examples, the producer uses the glass bottle 1305 with the FMC 115 for a premium retail product. The glass bottle format may be selected for shelf presentation. The FMC 115 includes the sealing member 1315 for the reverse-wedge seal on the glass bottle mouth, the umbrella valve 150, and the fluid well 125.

The threshold-based pressure relief of the umbrella valve 150 may reduce the risk of over-pressurization of actively fermenting beverages during distribution and storage. The semi-permeable membrane 170 may reduce the risk of spillage of the viewing fluid 135 during transport. The pressure-responsive bottle wall expansion may provide a passive visual indication to a retail consumer that the beverage is actively carbonated. The secondary seal 400 may reduce the risk of tampering with the FMC 115 during distribution.

An industrial beverage producer storing unpasteurized beverages in sealed containers faces a challenge: unpasteurized beverages may continue to ferment after filling and sealing, generating internal pressure during storage. A passive, threshold-based pressure-relief system integrated into a disposable cap may reduce the risk of over-pressurization during industrial storage without separate pressure-management hardware for each container.

In some examples, an industrial producer fills plastic bottles with an unpasteurized juice and installs a disposable FMC 115 on each bottle. The FMC 115 includes the umbrella valve 150 selected for a threshold pressure corresponding to the maximum safe internal pressure for the bottle format and storage conditions. The disposable FMC 115 is a single-use cap. After the storage period, the disposable FMC 115 is removed and replaced with a standard serving closure.

In another example, the producer uses the spunding valve adapter 905 on large-format storage vessels during fermentation pressure control. The FMC 115 is received in the spunding valve adapter 905. The umbrella valve 150 is selected for a threshold pressure corresponding to the vessel pressure rating. The spunding valve adapter 905 is coupled to the vessel through the container coupler 950.

In some examples, the producer uses a kit of umbrella valves with different threshold pressures to select the appropriate valve for different storage conditions, vessel pressure ratings, and beverage types. The same FMC 115 body is used across different storage applications by replacing the umbrella valve 150 with a valve of a different durometer or geometry.

The disposable FMC 115 may reduce the cost and complexity of pressure management for industrial storage of unpasteurized beverages by integrating pressure relief into a single-use cap. The selectable umbrella valve threshold may allow the same cap body to be used across different storage conditions and vessel pressure ratings. The spunding valve adapter 905 may allow the same umbrella valve platform to be applied to large-format storage vessels without separate pressure-management hardware.

In laboratory and medical contexts, sealed vessels containing biological cultures, fermentation media, and/or gas-generating chemical reactions may generate internal pressure during incubation or reaction. A passive, threshold-based pressure-relief system integrated into a vessel cap or adapter may reduce the risk of over-pressurization of sealed laboratory vessels without active monitoring or separate pressure-relief hardware.

In some examples, a laboratory researcher uses the FMC 115 on a sealed vessel containing a biological culture undergoing fermentation. The umbrella valve 150 is selected for a threshold pressure corresponding to the safe operating pressure of the vessel. When the internal pressure exceeds the threshold, the umbrella valve 150 opens transiently and releases gas. The umbrella valve 150 then closes. The researcher observes the gas bubbles 165 in the viewing fluid 135 as a passive indication that the internal pressure has reached the threshold.

In some examples, a laboratory uses the modular cap platform with a jar cap body configured to couple to a standard laboratory jar or vessel. The same umbrella valve 150 used in the FMC 115 is received in the jar cap body. The jar cap body defines a pressure pathway placing the umbrella valve 150 in fluid communication with the interior of the jar. The threshold pressure is selected by choosing an umbrella valve 150 of a corresponding durometer from a kit.

In some examples, the semi-permeable membrane 170 is used over the fluid well 125 to reduce the risk of spillage of the viewing fluid 135 during handling of the vessel in a laboratory environment.

The passive visual indication of the gas bubbles 165 may allow a researcher to observe pressure events in a sealed vessel without opening the vessel or using a separate pressure gauge. The selectable umbrella valve threshold may allow the same cap body to be used across vessels of different pressure ratings and biological cultures of different gas-generation rates. The rubber umbrella valve 150 failure mode—remaining open rather than sealing closed—may reduce the risk of over-pressurization of a sealed laboratory vessel.

In military and field environments, personnel may store, transport, and/or prepare fermented or carbonated fluids under conditions where access to specialized equipment is limited. A passive, threshold-based pressure-management system that operates without power, without electronic components, and without specialized tools may be applicable in field conditions.

In some examples, field personnel use the FMC 115 on a plastic bottle 1605 containing a fermentable beverage during field preparation of a probiotic or carbonated drink. The FMC 115 includes the umbrella valve 150 and the fluid well 125. The umbrella valve 150 is pre-selected for a threshold pressure corresponding to the target carbonation level and the pressure rating of the bottle. For example, the FMC 115 may advantageously be operated without a power source, electronic component, and/or specialized tool.

In some examples, field personnel use the pressure-responsive plastic bottle 1605 of FIG. 18. The visible expansion of the bottle wall provides a passive indication of internal pressure state, even without instrumentation.

In some examples, the FMC 115 is a disposable cap. After use, the disposable FMC 115 is discarded and replaced with a standard closure. The low cost and disposability of the FMC 115 may reduce the logistical burden of pressure management in field conditions.

The passive, non-electronic operation of the FMC 115 and the umbrella valve 150 may allow pressure management and visual indication in field conditions without power or specialized equipment. The disposable FMC 115 may reduce logistical burden. The pressure-responsive bottle wall expansion may provide a passive pressure indication without any cap-based indicator.

A consumer storing a partially fermented or carbonated beverage in a home refrigerator faces a challenge: the beverage may continue to ferment slowly at refrigerator temperatures, and internal pressure may build during storage. Tipping or jostling the bottle during refrigerator storage may risk spillage of the viewing fluid 135 from the fluid well 125.

In some examples, a consumer installs the FMC 115 with the semi-permeable membrane 170 over the fluid well 125 on a bottle of partially fermented beverage before placing the bottle in the refrigerator. The semi-permeable membrane 170 covers the fluid well 125. The semi-permeable membrane 170 is gas-permeable and liquid-impermeable. The viewing fluid 135 remains in the fluid well 125 even if the bottle is tipped during refrigerator storage. The consumer can observe the viewing fluid 135 through the semi-permeable membrane 170 when the membrane is at least partially transparent.

In an example, a consumer transports a bottle of fermenting beverage in a bag or cooler. The semi-permeable membrane 170 reduces the risk of spillage of the viewing fluid 135 during transport. The umbrella valve 150 continues to manage internal pressure during transport.

In some examples, the consumer uses the secondary seal 400 of FIG. 4 after fermentation is substantially complete to seal the bottle for long-term refrigerator storage. The secondary seal 400 seals the FMC 115 and the umbrella valve 150 against the outside environment.

The semi-permeable membrane 170 over the fluid well 125 may reduce the risk of spillage of the viewing fluid 135 during refrigerator storage and transport. The secondary seal 400 may allow long-term storage of the fermented beverage in the same bottle used for fermentation. The passive visual indication of the gas bubbles 165 may allow the consumer to monitor slow fermentation during refrigerator storage without opening the bottle.

A premium beverage producer or specialty retailer using glass bottle formats for product presentation faces a challenge: glass bottles present a different sealing surface than plastic bottles, and standard plastic caps with umbrella valves and fluid wells are designed for plastic bottle threads and finishes. A plastic cap with inner sealing fins configured to engage a tapered glass bottle mouth may allow the same umbrella valve and fluid well platform to be applied to glass bottle formats, such as without a separate cap design.

In some examples, a premium kombucha producer fills the glass bottle 1305 with an actively fermenting kombucha. The FMC 115 is installed on the glass bottle 1305. The sealing member 1315 of the FMC 115 engages the inner contour 1310 of the glass bottle mouth in a reverse-wedge sealing action. The FMC 115 includes the umbrella valve 150 and the fluid well 125. The glass bottle 1305 and the FMC 115 together define the enclosed chamber 155.

In another example, the producer uses the glass bottle 1305 with a hardened or reinforced base configured to resist warping under internal pressure during fermentation or carbonation. The hardened base may reduce the risk of the glass bottle 1305 deforming at the base under the internal pressures generated during fermentation.

In some examples, the producer uses the same FMC 115 on both the glass bottle 1305 and the plastic bottle 1605. The FMC 115 is directly receivable on both bottle formats. The same umbrella valve 150 is used in both applications.

The reverse-wedge sealing action of the sealing member 1315 against the inner contour 1310 may allow a plastic cap incorporating the umbrella valve and fluid well to form a pressure-resistant seal on a glass bottle without a separate elastomeric liner. The internal pressure acting on the sealing member 1315 may increase the sealing force at the plastic-on-glass interface. The common FMC 115 across glass and plastic bottle formats may reduce the number of different cap types a producer stocks. The glass bottle format may be selected for premium product presentation while retaining the same pressure-management and visual-indication platform used on plastic bottles.

An operator working with multiple vessel formats—plastic bottles, glass bottles, growlers, mini-kegs, and ball-lock-equipped vessels—across different stages of a fermentation or carbonation process would otherwise purchase separate, incompatible pressure-management solutions for each format. A modular cap platform using a common umbrella valve across all formats may reduce the number of different components the operator stocks and may allow consistent pressure-threshold behavior across all vessel formats.

In an example, an operator uses the following cap and adapter bodies from the modular platform: the FMC 115 on plastic bottles 1605; the FMC 115 on glass bottles 1305; the FMC 115 threaded directly onto ball-lock fittings 1105 on growlers; and the spunding valve adapter 905 on mini-kegs and corny-keg-style vessels. Each of these bodies receives the same umbrella valve 150. Each body defines a pressure pathway placing the umbrella valve 150 in fluid communication with the interior of the respective vessel.

The operator selects the umbrella valve 150 threshold for each application from a common kit of umbrella valves. The same kit is used across all vessel formats. The operator replaces the umbrella valve 150 in any body by removing the existing valve and inserting a replacement valve of a different durometer or geometry from the kit.

In another example, the operator uses a valve cartridge insert receivable in each of the cap and adapter bodies. The valve cartridge insert carries the umbrella valve 150 and defines the valve-receiving aperture. The same valve cartridge insert is receivable in the FMC 115 body, the spunding valve adapter 905 body, and the ball-lock adapter body. The operator may, for example, replace the valve cartridge insert independently of the host body, such as for cleaning, replacement, and/or threshold change.

The common umbrella valve 150 across all cap and adapter bodies may reduce the number of different valve types the operator stocks. The selectable threshold from a common kit may allow the operator to apply consistent pressure-threshold behavior across different vessel formats and applications. The valve cartridge insert may allow the umbrella valve to be replaced or cleaned independently of the host body. The modular platform may allow the operator to add new vessel formats to the system by adding a new cap or adapter body without changing the umbrella valve or the threshold-selection process.

In some implementations, the solid cap (e.g., cap 1320) and the FMC 115 with the umbrella valve 150 may be interchanged on vessel formats other than the glass bottle 1305. For example, the solid cap may be receivable on the plastic bottle 1605, on a growler, on a vessel coupled via the spunding valve adapter 905, and/or on a vessel coupled via the ball-lock 1105. In some examples, the solid cap may share a common coupling interface (e.g., a PCO 1881 thread or other standard thread) with the FMC 115, such that the solid cap and the FMC 115 are each receivable on any vessel having a corresponding threaded finish. In some implementations, a user may install the FMC 115 with the umbrella valve 150 and the fluid well 125 on a plastic bottle 1605 during an active fermentation or carbonation phase, and may subsequently replace the FMC 115 with the solid cap on the same plastic bottle 1605 for long-term sealed storage, transport, and/or serving. In some examples, the solid cap may be used on a growler or mini-keg after a fermentation or carbonation phase has been completed using the FMC 115 with the umbrella valve 150 on that same growler or mini-keg. In various implementations, the interchangeability of the solid cap and the FMC 115 across vessel formats may allow a user to apply threshold-based pressure management during active fermentation or carbonation using the FMC 115, and to subsequently apply a ventless sealed closure using the solid cap, such as, for example, such as without a different cap geometry and/or coupling interface for each vessel format.

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although an example of a system, which may be portable, has been described with reference to the above figures, other implementations may be deployed in other processing applications, such as desktop and networked environments.

Temporary auxiliary energy inputs may be received, for example, from chargeable or single use batteries, which may enable use in portable or remote applications. Some embodiments may operate with other DC voltage sources, such as a 9V (nominal) batteries, for example. Alternating current (AC) inputs, which may be provided, for example from a 50/60 Hz power port, or from a portable electric generator, may be received via a rectifier and appropriate scaling. Provision for AC (e.g., sine wave, square wave, triangular wave) inputs may include a line frequency transformer to provide voltage step-up, voltage step-down, and/or isolation.

Although particular features of an architecture have been described, other features may be incorporated to improve performance. For example, caching (e.g., L1, L2, . . . ) techniques may be used. Random access memory may be included, for example, to provide scratch pad memory and or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and, CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device, such as a CRT (cathode ray tube) or LCD (liquid crystal display) monitor for displaying information to the user, a keyboard, and a pointing device, such as a mouse or a trackball by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (e.g., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

As an illustrative example, an umbrella valve includes an umbrella valve body that extends along a longitudinal axis. The umbrella valve also includes a diaphragm that extends outwards away from the longitudinal axis and along the longitudinal axis towards a proximal end of the umbrella valve body. The umbrella valve also includes a retention module disposed on the umbrella valve body. The retention module is configured such that, when the umbrella valve body is disposed through an aperture in a surface such that the retention module is disposed at least partially on a first side of the surface and the umbrella valve is on an opposite side of the surface, the retention module engages the first side of the surface to resist the diaphragm retracting the umbrella valve body through the aperture. An outer circumference of the diaphragm defines a perimeter in a first plane. In a non-deployed mode, the retention module is resting above the first plane such that, in a deployed mode in which the retention module engages the first side of the surface, the umbrella valve is preloaded with a predetermined tension.

In some examples, the predetermined tension is configured at least by a hardness of the diaphragm.

In some examples, the perimeter of the diaphragm is a circle.

In some examples, the umbrella valve is provided together with a vessel cap.

In some examples, the vessel cap is threaded to close a container opening.

In some examples, the container is a glass bottle having an inner taper and the vessel cap includes a flexible fin configured to sealingly engage the inner taper.

In some examples, the umbrella valve and vessel cap are further provided with a spunding valve adapter in which the vessel cap and the umbrella valve act as a sole valve mechanism of the spunding valve adapter.

In some examples, the umbrella valve and vessel cap are further provided with a ball-lock adapter. The ball-lock adapter includes a lower body configured to close a container opening and threaded to receive the vessel cap that includes the umbrella valve. The ball-lock adapter also includes an upper body configured to sealingly capture the vessel cap with the umbrella valve between the upper body and the lower body such that the vessel cap and the umbrella valve act as a sole valve mechanism of the ball-lock adapter.

In some examples, the vessel cap includes a fluid well having a bottom surface that is the first side of the surface such that, in the non-deployed mode, the retention module is resting above the first plane such that, when the umbrella valve is coupled to the fluid well, the umbrella valve is preloaded with the predetermined tension.

In some examples, when the vessel cap is coupled to a vessel to close a chamber, the umbrella valve operates in a gas retention mode such that the umbrella valve substantially prevents a flowing communication between the chamber and the fluid well. When a gas pressure in the chamber exceeds a predetermined threshold, the umbrella valve operates in a gas release mode such that the umbrella valve allows the flowing communication between the chamber and the fluid well to be established. After the gas pressure is reduced below the predetermined threshold, the umbrella valve returns to the gas retention mode.

In some examples, the umbrella valve is replaceable with a second umbrella valve of a different hardness associated with a different predetermined tension corresponding to a user-selected predetermined threshold.

In some examples, the umbrella valve includes a cavity extending from a top surface of the umbrella valve towards an opposing end along the longitudinal axis.

In some examples, the cavity terminates above the first plane.

In some examples, the umbrella valve further includes a second plane above the first plane and below the retention module.

In some examples, the cavity terminates above the second plane.

In some examples, an outer periphery of the diaphragm includes a cylindrical surface concentric with and parallel to the longitudinal axis.

In some examples, the diaphragm includes an outwardly extending umbrella portion and a lip portion extending towards the proximal end. An inner surface of the lip portion is defined by an increasing radius relative to the longitudinal axis from an underside of the umbrella portion towards the proximal end.

In some examples, a thickness of the lip portion is greater than a thickness of the umbrella portion.

In some examples, the diaphragm is configured such that the outer periphery defines an outer periphery of the lip portion and does not flex to be parallel with the umbrella portion during a pressure release.

In some examples, the retention module includes a maximum proximal slope greater than a maximum distal slope.

As an illustrative example, a fermentation monitoring apparatus includes a vessel cap. The vessel cap includes a first chamber defining a fluid well configured to hold a quantity of fluid. The vessel cap also includes at least one fluid communication channel. The vessel cap also includes a stopping valve configured to substantially cover an aperture corresponding to each of the at least one fluid communication channel. The vessel cap is configured such that, when the vessel cap is coupled to a first end of a vessel to define a second chamber, the quantity of fluid is disposed in the fluid well, and a gas pressure in the second chamber exceeds a predetermined threshold, then the stopping valve permits gas to escape, through the at least one fluid communication channel, into the fluid well. The escaping gas forms bubbles in the quantity of fluid in the fluid well to provide visual indication to a user that the gas has exceeded the predetermined threshold.

In some examples, the fermentation monitoring apparatus further includes a cover configured to substantially cover the fluid well. The cover includes a selectively permeable membrane such that a liquid content in the fluid well is prevented from escaping the fluid well, and gaseous content in the fluid well is permitted to escape the fluid well through the selectively permeable membrane.

In some examples, the fermentation monitoring apparatus further includes a crown cap configured to be applied to substantially cover the fluid well after a fermentation process. When the crown cap is applied, the crown cap substantially seals the vessel cap to prevent fluid communication between the second chamber and an exterior of the vessel via the at least one fluid communication channel and the fluid well.

In some examples, the fermentation monitoring apparatus further includes a tasting lid releasably coupled to the vessel cap in sealing occlusion of a second fluid communication channel between an exterior of the vessel cap and the second chamber. The tasting lid is configured such that, when the tasting lid is removed, fluid communication is established to contents of the second chamber via the second fluid communication channel.

In some examples, the fermentation monitoring apparatus further includes a residue collection chamber releasably coupled to a second end of the vessel to selectively collect residue from the second chamber.

In some examples, the residue collection chamber further includes a false bottom configured to selectively allow a residue in the chamber to flow into the chamber. The residue collection chamber also includes a residue valve selectively substantially preventing fluid flow between the chamber and the residue collection chamber such that, when the gas pressure in the chamber exceeds the predetermined threshold, the residue valve stops to prevent a residue from flowing into the chamber and the residue is collected in the residue collection chamber.

In some examples, the fermentation monitoring apparatus further includes a starter kit. The starter kit includes a quantity of fermentation catalyst and content for fermentation.

In some examples, the fermentation monitoring apparatus further includes at least one sensor and a wireless communication module configured such that data received from the sensor is transmitted to a remote device.

In some examples, the at least one sensor includes a temperature sensor.

As an illustrative example, a fermentation monitoring apparatus includes a vessel cap. The vessel cap includes a fluid well holding a quantity of fluid. The vessel cap also includes at least one fluid communication channel configured such that, when the vessel cap is coupled to a first end of a vessel to define a chamber and a gas pressure in the chamber exceeds a predetermined threshold, then the fluid well receives gas released from the channel via the at least one fluid communication channel such that a visual indicium of gas bubbles is formed in the quantity of fluid indicating that the gas pressure in the chamber has exceeded the predetermined threshold.

In some examples, the fermentation monitoring apparatus further includes a stopping valve configured to substantially cover an aperture corresponding to each of the at least one fluid communication channel and prevent fluid communication between the chamber and the fluid well. When the gas pressure in the chamber exceeds the predetermined threshold, the stopping valve opens such that the fluid communication between the chamber and the fluid well is established.

In some examples, the fluid well includes a cover of selectively permeable membrane such that a selected content in the vessel is prevented from flowing out of the vessel, and other content in the vessel is allowed to traverse through the selectively permeable membrane.

In some examples, the fermentation monitoring apparatus further includes a crown cap to be applied after a fermentation process. The crown cap substantially encloses the vessel cap to preserve a fermented content in the chamber.

In some examples, the fermentation monitoring apparatus further includes a tasting lid releasably coupled to the chamber such that, when the tasting lid is removed, a flowing channel is established to content within the channel.

In some examples, the fermentation monitoring apparatus further includes a residue collection chamber releasably coupled to a second end of the vessel to selectively collect residue from the chamber.

In some examples, the residue collection chamber includes a false bottom that selectively allows a residue in the chamber to flow into the chamber. The residue collection chamber also includes a residue valve substantially preventing a flow channel between the chamber and the residue collection chamber such that, when the gas pressure in the chamber exceeds the predetermined threshold, the residue valve stops to prevent a residue from flowing into the chamber and the residue is collected in the residue collection chamber.

In some examples, the fermentation monitoring apparatus further includes a starter kit. The starter kit includes a quantity of fermentation catalyst and content for fermentation.

As an illustrative example, a vessel cap includes a fluid well. The vessel cap also includes an umbrella valve having a diaphragm and a retention module. An outer circumference of the diaphragm defines a circle in a first plane. In a non-deployed mode, the retention module is resting above the first plane such that, when the umbrella valve is coupled to the fluid well, the umbrella valve is preloaded with a predetermined tension.

In some examples, when the vessel cap is coupled to a first end of a vessel to define a chamber, the umbrella valve operates in a gas retention mode such that the umbrella valve substantially prevents a flowing communication between the chamber and the fluid well. When a gas pressure in the chamber exceeds a predetermined threshold, the umbrella valve operates in a gas release mode such that the umbrella valve allows the flowing communication between the chamber and the fluid well to be established. After the gas pressure is reduced below the predetermined threshold, the umbrella valve returns to the gas retention mode.

In some examples, the umbrella valve is replaceable with an umbrella valve of different hardness associated with a different predetermined tension corresponding to a user-selected predetermined threshold.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. An umbrella valve, comprising:
an umbrella valve body (150) extending along a longitudinal axis;
a diaphragm (225) extending outwards away from the longitudinal axis and along the longitudinal axis towards a proximal end of the umbrella valve body; and,
a retention module (220) disposed on the umbrella valve body and configured such that, when the umbrella valve body is disposed through an aperture in a surface such that the retention module is disposed at least partially on a first side of the surface and the umbrella valve is on an opposite side of the surface, the retention module engages the first side of the surface to resist the diaphragm retracting the umbrella valve body through the aperture,
wherein:
an outer circumference of the diaphragm defines a perimeter in a first plane, and, in a non-deployed mode, the retention module is resting above the first plane such that, in a deployed mode in which the retention module engages the first side of the surface, the umbrella valve is preloaded with a predetermined tension.

2. The umbrella valve of claim 1, wherein the predetermined tension is configured at least by a hardness of the diaphragm.

3. The umbrella valve of claim 1, wherein the perimeter of the diaphragm is a circle.

4. The umbrella valve of claim 1 together with a vessel cap.

5. The umbrella valve and vessel cap of claim 4, wherein the vessel cap is threaded to close a container opening.

6. The umbrella valve and vessel cap of claim 5, wherein the container is a glass bottle with an inner taper and the vessel cap comprises a flexible fin configured to sealingly engage the inner taper.

7. The umbrella valve and vessel cap of claim 5, further comprising a spunding valve in which the vessel cap and the umbrella valve act as a sole valve mechanism of the spunding valve.

8. The umbrella valve of claim 5, further comprising a ball-lock adapter comprising a lower body configured to close a container opening and threaded to receive the vessel cap comprising the umbrella valve, the ball-lock adapter comprising an upper body configured to sealingly capture the vessel cap with the umbrella valve between the upper body and the lower body such that the vessel cap and umbrella valve act as a sole valve mechanism of the ball-lock adapter.

9. The umbrella valve and vessel cap of claim 4, wherein:
the vessel cap comprises a fluid well (125) with a bottom surface comprising the first side the surface such that, in a non-deployed mode, the retention module is resting above the first plane such that, when the umbrella valve is coupled to the fluid well, the umbrella valve is preloaded with a predetermined tension.

10. The umbrella valve and vessel cap of claim 9, wherein, when the vessel cap is coupled to a vessel to close a chamber,
the umbrella valve operates in a gas retention mode such that the umbrella valve substantially prevents a flowing communication between the chamber and the fluid well,
when a gas pressure in the chamber exceeds a predetermined threshold, the umbrella valve operates in a gas release mode such that the umbrella valve allows the flowing communication between the chamber and the fluid well to be established, and
after the gas pressure is reduced below the predetermined threshold, the umbrella valve returns to the gas retention mode.

11. The umbrella valve and vessel cap of claim 10, wherein the umbrella valve is replaceable with an umbrella valve of different hardness associated with a different predetermined tension corresponding to a user-selected predetermined threshold.

12. The umbrella valve of claim 1, comprising a cavity from a top surface of the umbrella valve extending towards an opposing end along the longitudinal axis.

13. The umbrella valve of claim 12, wherein the cavity terminates above the first plane.

14. The umbrella valve of claim 13, further comprising a second plane above the first plane and below the retention module.

15. The umbrella valve of claim 14, wherein the cavity terminates above the second plane.

16. The umbrella valve of claim 1, wherein an outer periphery of the diaphragm comprises a cylindrical surface concentric with and parallel to the longitudinal axis.

17. The umbrella valve of claim 16, wherein the diaphragm comprises an outwardly extending umbrella portion and a lip portion extending towards the proximal end, and wherein an inner surface of the lip portion is defined by an increasing radius relative to the longitudinal axis from an underside of the umbrella portion towards the proximal end.

18. The umbrella valve of claim 17, wherein a thickness of the lip portion is greater than a thickness of the umbrella portion.

19. The umbrella valve of claim 18, wherein the diaphragm is configured such that the outer periphery defines an outer periphery of the lip portion and does not flex to parallel with the umbrella portion during a pressure release.

20. The umbrella valve of claim 1, wherein the retention module comprises a maximum proximal slope greater than a maximum distal slope.
